(19) United States
(12) Patent Application Publication
(10) Pub. No.: US 2023/0414783 A1
(43) Pub. Date: Dec. 28, 2023

(54) Biliary Delivery Methods, Compositions and Kits for Use Therein

(71) Applicant: AMBYS MEDICINES, INC., South San Francisco, CA (US)

(72) Inventors: Whitney Krey, Rochester, MN (US); Garrett Heffner, Rochester, MN (US)

(21) Appl. No.: 18/265,171

(22) PCT Filed: Dec. 20, 2021
(86) PCT No.: PCT/US2021/064359

§ 371 Date: Jun. 2, 2023

Related U.S. Application Data
(60) Provisional application No. 63/236,574, filed on Aug. 24, 2021.
(60) Provisional application No. 63/175,693, filed on Apr. 16, 2021.
(60) Provisional application No. 63/128,983, filed on Dec. 22, 2020.

(51) Int. Cl. A61K 48/00; C12N 15/86; A61K 31/785; A61P 1/16

(52) U.S. Cl. CPC A61K 48/0041; C12N 15/86; A61K 31/785; A61K 48/0075; A61P 1/16; C12N 2740/15043; C12N 2740/15045

(57) ABSTRACT

The present disclosure provides composition and methods for delivery of therapeutic biologics to locations where bile is present, including the biliary tract, and compositions and kits for use in such methods. Also provided are compositions that include, and methods that employ, biliary-therapeutic enhancers for the delivery of therapeutic biologics. Methods of biliary tract delivery of a therapeutic biologic, as described herein, generally include one or more administrations of the relevant biologic and biliary-therapeutic enhancer Biliary delivery compositions will generally include one or more biliary-therapeutic enhancers and a therapeutic biologic. As compared to the decreased activity of the therapeutic biologic in the presence of bile, the compositions, methods, and kits of the present disclosure result in increased, enhanced and/or rescued activity of the therapeutic in the presence of bile. Use of biliary-therapeutic enhancers, e.g., in the methods, compositions and kits described, are also provided.

### CROSS-REFERENCE TO RELATED APPLICATIONS

This application claims the benefit of U.S. Provisional Patent Application No. 63/236,574, filed Aug. 24, 2021, U.S. Provisional Patent Application No. 63/175,693, filed Apr. 16, 2021, and U.S. Provisional Patent Application No. 63/128,983, filed Dec. 22, 2020, which applications are incorporated herein by reference in their entireties.

### BACKGROUND

There are over 100,000 patients with acute liver disease and over half a million patients with decompensated liver cirrhosis in the United States alone. Liver disease accounts for 62,000 deaths annually in the US and approximately 2 million deaths worldwide, with 1.3 million due to cirrhosis specifically. As of 2019, cirrhosis was the 11th most common cause of death globally and the 12th leading cause in the US. Worldwide about 2 billion people consume alcohol, another approximately 2 billion adults are obese or overweight, and 400 million adults have diabetes. Alcohol consumption, liver lipid deposition, and insulin resistance are all considered to be major risk factors in the development of fibrosis and eventually cirrhosis. Moreover, while drug-induced liver injury continues to increase as a major cause of acute hepatitis, the global prevalence of viral hepatitis remains high.

Liver transplantation, when available and successful, is a life changing therapy that represents the second most common solid organ transplantation. However, suitable livers are often not available in needed quantities or in time for subjects with rapidly declining conditions such as acute liver failure. In comparison to the expansive disease prevalences described above, under 9,000 liver transplantations were performed in the US.

While many other therapies, including numerous biologics, have shown promise, either mechanistically or in preclinical studies, few have advanced through clinical trials or displayed significant positive outcomes in human subjects. For example, targeting collagen crosslinking, an essential process in fibrosis, through monoclonal antibody inhibition of lysyl oxidase (LOX) family enzymes indicated potential in in vitro and preclinical studies (see e.g., Ikenaga et al. Gut (2017) 66:1697-1708). However, clinical testing of selonsertib, administered either by subcutaneous injection or intravenous infusion, did not demonstrate clinical efficacy (Harrison et al. Gastroenterology (2018) 155(4):1140-1153). Other therapeutics directed at liver diseases, or pathological characteristics thereof such as fibrosis and cirrhosis, have illustrated a similar pattern of early promise followed by unsatisfactory clinical outcomes.

### SUMMARY

The present disclosure provides methods for delivery of therapeutic biologics to locations where bile is present, including the biliary tract, and compositions and kits for use in such methods. Also provided are compositions (e.g., pharmaceutical compositions) that include, and methods that employ, biliary-therapeutic enhancers for the delivery of therapeutic biologics. The biliary-delivery compositions (e.g., pharmaceutical compositions) include one or more therapeutic biologics and/or one or more biliary-therapeutic enhancers (e.g., sequestrants), which may be included in separate compositions and/or combined into a single composition (e.g., pharmaceutical composition). Methods of biliary tract delivery of a therapeutic biologic, as described herein, generally include one or more administrations of the relevant biologic, biliary-therapeutic enhancer and/or composition as described herein. As compared to the decreased activity of the therapeutic biologic in the presence of bile, the compositions, methods, and kits of the present disclosure result in surprisingly and unexpectedly increased, enhanced and/or rescued activity of the therapeutic in the presence of bile. Use of biliary-therapeutic enhancers, e.g., in the methods, compositions and kits described, are also provided.

In some aspects, described herein are compositions comprising one or more therapeutic biologics and/or one or more biliary-therapeutic enhancers (e.g., one or more sequestrants and/or one or more transduction enhancers). The therapeutic biologic(s) and/or biliary-therapeutic enhancer(s) may be formulated in the same or in one or more different compositions (e.g., one or more separate compositions comprising one or more therapeutic biologicals and one or more separate compositions comprising one or more biliary-therapeutic enhancer(s) or one or more combination compositions comprising one or more therapeutic biologicals and one or more biliary-therapeutic enhancers). In some embodiments, the composition comprises a combination of at least one therapeutic biologic and at least one biliary-therapeutic enhancer. In certain embodiments, the at least one biliary-therapeutic enhancer of the composition comprises at least one bile sequestrant. In other embodiments, the at least one biliary-therapeutic enhancer comprises a transduction enhancer. In still further embodiments, the composition comprises a combination of at least one least one sequestrant and at least one transduction enhancer. The compositions of the invention may be pharmaceutical compositions and may further comprise one or more pharmaceutically acceptable excipients, buffers, and/or other reagents. Any amount (dosage) of therapeutic biologic and biliary therapeutic enhancer may be used in the separate or combined compositions described herein. Useful dosages of therapeutic biologic and/or biliary therapeutic enhancer, administered separately or together, may be readily determined.

Thus, aspects of the present disclosure also include pharmaceutical compositions, including where such compositions include: a pharmaceutically acceptable carrier configured as a liquid for delivery to the biliary tract; an effective amount of one or more therapeutic biologics; and one or more biliary-therapeutic enhancers. The pharmaceutical compositions (formulations) may include one or more of biliary-therapeutic enhancers in the same or different formulations from the therapeutic biologicals. In certain embodiments, the one or more biliary-therapeutic enhancers may be formulated with additional components (e.g., with the one or more therapeutic biologics). In other embodiments, separate pharmaceutical compositions (formulations) of any of the components may also be employed, for example the one or more therapeutic biologics may be formulated in one or more different formulations than the one or more biliary-therapeutic enhancers.

In some aspects the compositions may include a therapeutic biologic that is liable to inactivation by bile. In some aspects, the therapeutic biologic of the composition may include, without limitation, a gene therapy agent (e.g., a nucleic acid construct encoding one or more therapeutic proteins or a portion thereof, a non-coding nucleic acid, etc.), and/or a protein, including without limitation, a nonviral vector, a viral vector, a lipid nanoparticle (LNP), an enveloped viral vector, a lentiviral vector, an adenovirus, an adeno-associated virus (AAV) vector, a therapeutic peptide, and/or an antibody. In certain embodiments, the viral (e.g., lentiviral or AAV) or non-viral vector comprises one or more polynucleotides encoding one or more proteins (e.g., therapeutic proteins, antibodies or the like) including but not limited to proteins described herein, including e.g., proteins that are lacking (i.e., deficient and/or absent) in liver disease.

In some aspects the biliary therapeutic enhancer of the composition may include, without limitation, a polyamine or polyether polymer. In some instances, the biliary therapeutic enhancer comprises one or more bile acid sequestrants, including but not limited to, e.g., colesevelam, colestyramine, colestipol, and sevelamer. In some instances, the biliary therapeutic enhancer may be a cationic or nonionic amphiphilic transduction enhancer, including but not limited to, e.g., polybrene, protamine sulfate, polyethyleneimine (PEI), Poly(ethylene glycol) (PEG), poly-L-lysine, and F108. Combinations of any biliary therapeutic enhancers (e.g., one or more sequestrants and/or one or more transduction enhancers) may be used.

Aspects of the present disclosure also include use of a liquid composition comprising a biliary-therapeutic enhancer for delivery of a therapeutic biologic to the biliary tract of a subject to treat the subject for a liver condition. In some aspects, the biliary-therapeutic enhancer of such a use is a bile acid sequestrant, optionally wherein the bile acid sequestrant is a polyamine or polyether polymer. In some aspects, the biliary-therapeutic enhancer of such a use is a cationic or nonionic amphiphilic transduction enhancer, optionally wherein the cationic or nonionic amphiphilic transduction enhancer is a polyamine or polyether polymer.

A bile acid secretion inhibitor may also be included in a separate composition, and/or with a composition comprising the one or more therapeutic biologics and/or biliary-therapeutic enhancers. Bile acid secretion inhibitors include but are not limited to agonists of the bile acid receptor (BAR), also known as farnesoid X receptor (FXR) or NR1H4 (nuclear receptor subfamily 1, group H, member 4). In certain embodiments, the bile acid secretion inhibitor is an FXR agonist.

Aspects of the present disclosure include methods of treating a subject for a condition that include administering directly to the biliary tract of the subject an effective amount of one or more therapeutic biologics and an effective amount of one or more biliary-therapeutic enhancers, for example using one or more compositions as described herein (compositions comprising the therapeutic biologic(s) and/or biliary-therapeutic enhancer(s)) thereby treating the subject for the condition.

In some aspects, the condition treated by the methods and compositions described herein may be, without limitation, an inherited condition, a monogenic disease, a metabolic disease, or a liver condition. Subject conditions may include e.g., acute intermittent porphyria, acute liver failure, alagille syndrome, alcoholic fatty liver disease, alcoholic hepatitis, alcoholic liver cirrhosis, alcoholic liver disease, alpha 1-antitrypsin deficiency, amebic liver abscess, autoimmune hepatitis, biliary liver cirrhosis, budd-chiari syndrome, chemical and drug induced liver injury, cholestasis, chronic hepatitis, chronic hepatitis B, chronic hepatitis C, chronic hepatitis D, end stage liver disease, erythropoietic protoporphyria, fascioliasis, fatty liver disease, focal nodular hyperplasia, hepatic echinococcosis, hepatic encephalopathy, hepatic infarction, hepatic insufficiency, hepatic porphyrias, hepatic tuberculosis, hepatic veno-occlusive disease, hepatitis, hepatocellular carcinoma, hepatoerythropoietic porphyria, hepatolenticular degeneration, hepatomegaly, hepatopulmonary syndrome, hepatorenal syndrome, hereditary coproporphyria, liver abscess, liver cell adenoma, liver cirrhosis, liver failure, liver neoplasm, massive hepatic necrosis, non-alcoholic fatty liver disease, parasitic liver disease, peliosis hepatis, porphyria cutanea tarda, portal hypertension, pyogenic liver abscess, reye syndrome, variegate porphyria, viral hepatitis, viral hepatitis A, viral hepatitis B, viral hepatitis C, viral hepatitis D, viral hepatitis E, zellweger syndrome, and the like. In some aspects the condition may be a condition other than a hyperlipidemia, a secondary dyslipidemia, a bile acid malabsorption condition, and/or a diabetic condition.

In some aspects, retroductal delivery of the therapeutic biologic, biliary-therapeutic enhancer and/or compositions described herein is employed to deliver the therapeutic to the liver of the subject. In some instances, direct retroductal delivery of the therapeutic biologic results in an enhanced local hepatic concentration of the therapeutic biologic. In some instances, the therapeutic biologic is subject (liable) to inactivation by bile.

In some aspects of the methods the therapeutic biologic may include, without limitation, a gene therapy agent or a protein, including without limitation, a nonviral vector, a viral vector, a lipid nanoparticle, an enveloped viral vector, a lentiviral vector, an adenovirus, an adeno-associated virus (AAV), a therapeutic peptide, or an antibody (i.e., deficient and/or absent) in a subject with liver disease.

In some aspects, the biliary therapeutic enhancer may include, without limitation, a polyamine and/or polyether polymer. In some instances, the biliary therapeutic enhancer may be a bile acid sequestrant, including but not limited to, e.g., colesevelam, colestyramine, colestipol, and/or sevelamer. In some instances, the biliary therapeutic enhancer may be a cationic or nonionic amphiphilic transduction enhancer, including but not limited to, e.g., polybrene, protamine sulfate, polyethyleneimine (PEI), Poly(ethylene glycol) (PEG), poly-L-lysine, and/or F108.

In some aspects, the therapeutic biologic and the biliary-therapeutic enhancer may be co-administered. In some aspects of the methods the therapeutic biologic and the biliary-therapeutic enhancer may be co-formulated in a single pharmaceutical composition. In some aspects of the methods the biliary-therapeutic enhancer may be administered before the therapeutic biologic. In some aspects of the methods two or more biliary-therapeutic enhancers may be administered, including e.g., where the two or more biliary-therapeutic enhancers include a bile acid sequestrant and a cationic or nonionic amphiphilic transduction enhancer. In some aspects of the methods where two or more biliary-therapeutic enhancers may be administered, the two or more biliary-therapeutic enhancers may be co-administered or co-formulated in a single pharmaceutical composition.

In some aspects the methods may further include administering to the subject a bile acid secretion inhibitor, including but not limited to e.g., where the bile acid secretion inhibitor is an FXR agonist, optionally wherein the FXR agonist is selected from obeticholic acid or an FGF19 analog.

Aspects of the present disclosure also include kits that may include any of the compositions and/or reagents described herein, including e.g., a liquid pharmaceutically acceptable carrier; a therapeutic biologic; and/or a biliary-therapeutic enhancer. The kits may further comprise instructions for using the compositions and/or performing the methods described herein.

In some aspects, the biliary-therapeutic enhancer of the kit and the liquid pharmaceutically acceptable carrier of the kit are formulated together in a vessel of the kit. In some aspects the therapeutic biologic of the kit is liable to inactivation by bile. In some aspects, the therapeutic biologic of the kit may include, without limitation, a gene therapy agent or a protein, including without limitation, a nonviral vector, a viral vector, a lipid nanoparticle, an enveloped viral vector, a lentiviral vector, an adenovirus, an adeno-associated virus (AAV), a therapeutic peptide, or an antibody. In certain embodiments, the viral or non-viral vector comprises one or more polynucleotides encoding one or more proteins (e.g., therapeutic proteins, antibodies or the like), for example one or more proteins as described herein lacking (i.e., deficient and/or absent) in one or more liver conditions.

In some aspects the biliary therapeutic enhancer of the kit may include, without limitation, a polyamine or polyether polymer. In some instances, the biliary therapeutic enhancer may be a bile acid sequestrant, including but not limited to, e.g., colesevelam, colestyramine, colestipol, and sevelamer. In some instances, the biliary therapeutic enhancer may be a cationic or nonionic amphiphilic transduction enhancer, including but not limited to, e.g., polybrene, protamine sulfate, polyethyleneimine (PEI), Poly(ethylene glycol) (PEG), poly-L-lysine, and F108.

In some aspects the kit may include a device, including without limitation where the device is configured for retroductal delivery of the therapeutic biologic to the liver. In some aspects the kit may be used for the treatment of a condition described herein.

Aspects of the present disclosure include methods of transducing or transfecting a cell that include contacting the cell in the presence of bile with a biliary-transduction enhancer to generate a transduction/transfection composition; and contacting the transduction/transfection composition with a gene therapy agent comprising an exogenous nucleic acid under conditions sufficient for transduction or transfection of the exogenous nucleic into the cell, thereby transducing or transfecting the cell with the exogenous nucleic acid.

In some aspects, the cell is a liver cell, optionally a hepatocyte. In some aspects, the gene therapy agent may include a nonviral vector or a viral vector, including a lipid nanoparticle or an enveloped viral vector. In some aspects, the viral vector is a lentiviral vector, an adenovirus vector, or an adeno-associated virus (AAV) vector.

In some aspects, the exogenous nucleic acid includes a coding sequence, including wherein the coding sequence encodes a transcription factor, a therapeutic peptide, or an antibody. In some aspects, the biliary-therapeutic enhancer comprises a polyamine or polyether polymer. In some aspects, the biliary-therapeutic enhancer is a bile acid sequestrant, such as colesevelam, colestyramine, colestipol, or sevelamer. In some aspects, the biliary-therapeutic enhancer is a cationic or nonionic amphiphilic transduction enhancer, such as polybrene, protamine sulfate, polyethyleneimine (PEI), Poly(ethylene glycol) (PEG), poly-L-lysine, or F108. In some aspects, contacting the cell with the biliary-transduction enhancer may include contacting the cell with both a bile acid sequestrant and a cationic or nonionic amphiphilic transduction enhancer. In some aspects, the biliary-transduction enhancer and the gene therapy agent are present together in a formulation prior to the contacting.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1 is a graph showing results from lentivirus transduction assays in HeLaRC32 cells demonstrating that the addition of bile acid sequestrants to transduction reactions rescues the activity of lentivirus vector (“LV-SFFV-LUC2-P2A-EmGFP” comprising luciferase and Emerald GFP transgenes linked via a 2A peptide driven by a spleen focus forming virus (SFFV) promoter) as measured by relative luciferase units (“RLU”) that is otherwise substantially reduced in the presence of bile.

FIG. 2 is a graph showing results from lentivirus transduction assays performed in a series of bile dilutions in the presence of different transduction enhancers (polyethyleneimine “PEI” or polyethylene glycol) demonstrating that transduction enhancers alone can rescue lentiviral vector activity otherwise lost in the presence of bile.

FIG. 3 is a graph showing results from a bile time series demonstrating the temporal dynamics of inactivation of lentiviral vector (“LVV”) by exposure to 15% rat bile.

FIG. 4A and FIG. 4B are graphs showing the effect of bile sequestrants and/or enhancers on transduction efficiency of LNPs carrying mRNA encoding a green fluorescent protein marker (GFP). FIG. 4A shows the percent of cells expressing GFP at indicated concentrations of sequestrant (colesevelam) or transduction enhancer (F108). FIG. 4B is a graph showing the results of FIG. 4A in log format. “Bile” refers to samples including of 30% rat bile alone; “Bile+colesevelam” refers to samples containing bile and colesevelam (at the indicated concentrations); “Bile+F108” refers to samples containing bile and F108; and “No treatment” refers to controls not including bile, a sequestrant or an enhancer.

FIG. 5A and FIG. 5B show representative luciferase-stained liver sections from animals treated with vehicle or lentiviral vector (LVV) carrying a luciferase transgene. FIG. 5A shows liver sections from vehicle treated animals FIG. 5B shows sections from the LVV treated animals. As shown, only the LVV treated animals (FIG. 5B) expressed luciferase in their livers in vivo.

### DETAILED DESCRIPTION

The present disclosure provides composition and methods for enhanced delivery of therapeutic biologics to locations where bile is present, including the biliary tract, and compositions and kits for use in such methods. Also provided are compositions that include, and methods that employ, biliary-therapeutic enhancers for the delivery of therapeutic biologics. Methods of biliary tract delivery of a therapeutic biologic, as described herein, generally include one or more administrations of the relevant biologic and biliary-therapeutic enhancer. Biliary delivery compositions will generally include one or more biliary-therapeutic enhancers and a therapeutic biologic. As compared to the decreased activity of the therapeutic biologic in the presence of bile, the compositions, methods, and kits of the present disclosure result in increased, enhanced and/or rescued activity of the therapeutic in the presence of bile. Use of biliary-therapeutic enhancers, e.g., in the methods, compositions and kits described, are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Definitions

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a,” “an,” or “the” include plural referents, unless the context clearly dictates otherwise. Thus, for example, reference to “a cell” includes a plurality of such cells and reference to “the agent” includes reference to one or more agents known to those skilled in the art, and so forth.

The term “about” in relation to a reference numerical value can include a range of values plus or minus 10% from that value. For example, the amount “about 10” includes values from 9 to 11, including the values of 9, 10, and 11. The term “about” in relation to a reference numerical value can also include a range of values plus or minus 10%, 9%, 8%, 7%, 6%, 5%, 4%, 3%, 2%, or 1% from that value.

Before describing specific embodiments of the disclosure, it will be helpful to set forth definitions that are used in describing the present disclosure.

The term “biliary-therapeutic enhancer” refers to any compound, molecule or formulation of one or more compounds and molecules that enhances delivery, in the presence of bile, of a biologic to one or more liver cells. The liver cells may, without limitation, be isolated (e.g., isolated populations of mature or hepatic stem cells), in a living subject (in vivo as a whole liver), or may be obtained from a subject and introduced into the same or different subject (ex vivo). The term includes, but is not limited to, one or more bile sequestrants (also referred to as “sequestrants”) and/or one or more transduction enhancers, which may be administered concurrently (in the same or different formulations) or sequentially in any order (in the same or different formulations).

The term “assessing” includes any form of measurement, and includes determining if an element is present or not. The terms “determining”, “measuring”, “evaluating”, “assessing” and “assaying” are used interchangeably and include quantitative and qualitative determinations. Assessing may be relative or absolute.

The terms “control”, “control assay”, “control sample” and the like, refer to a sample, test, or other portion of an experimental or diagnostic procedure or experimental design for which an expected result is known with high certainty, e.g., in order to indicate whether the results obtained from associated experimental samples are reliable, indicate to what degree of confidence associated experimental results indicate a true result, and/or to allow for the calibration of experimental results. For example, in some instances, a control may be a “negative control” assay such that an essential component of the assay is excluded such that an experimenter may have high certainty that the negative control assay will not produce a positive result. In some instances, a control may be “positive control” such that all components of a particular assay are characterized and known, when combined, to produce a particular result in the assay being performed such that an experimenter may have high certainty that the positive control assay will not produce a positive result. Controls may also include “blank” samples, “standard” samples (e.g., “gold standard” samples), validated samples, etc.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a KD (dissociation constant) of 10⁵ M or less (e.g., 10⁶ M or less, 10⁷ M or less, 10⁸ M or less, 10⁹ M or less, 10¹⁰ M or less, 10¹¹ M or less, 10¹² M or less, 10″³ M or less, 10¹⁴ M or less, 10¹⁵ M or less, or 10¹⁶ M or less). “Affinity” refers to the strength of binding, increased binding affinity being correlated with a lower KD.

The terms “recipient”, “individual”, “subject”, “host”, and “patient”, are used interchangeably herein and refer to any mammalian subject for whom diagnosis, treatment, or therapy is desired, such as human subjects. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human. In some cases, the methods of the invention find use in experimental animals, in veterinary application, and/or in the development of animal models, including, but not limited to, rodents including mice, rats, and hamsters; rabbits, dogs, cats, non-human primates, and other animals.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. For example, a preventative treatment, i.e. a prophylactic treatment, may include a treatment that effectively prevents a condition (e.g., a liver condition) or a treatment that effectively prevents or controls progression of a condition (e.g., a liver condition). In some instances, the treatment may result in a treatment response, such as a complete response or a partial response. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s).

Those in need of treatment can include those already afflicted (e.g., those with a liver condition (e.g., acute liver condition, chronic liver condition, etc.), those with cirrhosis, those with fibrosis, etc.) as well as those in which prevention is desired (e.g., those with increased susceptibility to a liver condition; those suspected of having a liver condition; those with an increased risk of developing a liver condition; those with increased environmental exposure to practices or agents causing a liver condition; those suspected of having a genetic or behavioral predisposition to a liver condition; those with a liver condition; those having results from screening indicating an increased risk of a liver condition; those having tested positive for a liver condition; those having tested positive for one or more biomarkers of a liver condition, etc.).

A therapeutic treatment is one in which the subject is afflicted prior to administration and a prophylactic treatment is one in which the subject is not afflicted prior to administration. In some embodiments, the subject has an increased likelihood of becoming afflicted or is suspected of having an increased likelihood of becoming afflicted (e.g., relative to a standard, e.g., relative to the average individual, e.g., a subject may have a genetic predisposition to a condition and/or a family history indicating increased risk), in which case the treatment can be a prophylactic treatment.

As used herein the term “small molecule” refers to a small organic or inorganic compound having a molecular weight of more than 50 and less than about 2,500 daltons. Agents may comprise functional groups necessary for structural interaction with proteins, particularly hydrogen bonding, and may include at least an amine, carbonyl, hydroxyl or carboxyl group, and may contain at least two of the functional chemical groups. The small molecule agents may comprise cyclical carbon or heterocyclic structures and/or aromatic or polyaromatic structures substituted with one or more functional groups. Small molecule agents are also found among biomolecules including peptides, saccharides, fatty acids, steroids, purines, pyrimidines, derivatives, structural analogs or combinations thereof.

The term “recombinant”, as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide, means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

The terms “nucleic acid” and “polynucleotide” as used interchangeably herein refer to a polymeric form of nucleotides of any length, either ribonucleotides or deoxyribonucleotides, including analogs thereof. The terms refer only to the primary structure of the molecule. Thus, this term includes double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. Non-limiting examples of nucleic acids and polynucleotides include linear and circular nucleic acids, messenger RNA (mRNA), cDNA, recombinant polynucleotides, vectors, probes, primers, single-, double-, or multi-stranded DNA or RNA, genomic DNA, DNA-RNA hybrids, chemically or biochemically modified, non-natural, or derivatized nucleotide bases, oligonucleotides containing modified or non-natural nucleotide bases (e.g., locked-nucleic acids (LNA) oligonucleotides), and interfering RNAs. In some instances, a polynucleotide may be a continuous open reading frame polynucleotide that excludes at least some non-coding sequence from a corresponding sequence present in the genome of an organism.

The term “nucleic acid therapeutic”, as used herein, generally refers to nucleic acid that may be administered in a therapeutic context to treat a subject for a condition, e.g., as applied in the context of gene therapy as described herein. Non-limiting examples of nucleic acid therapeutics include interfering nucleic acids for repressing expression of a gene associated with a condition, a coding sequence (e.g., arranged in an expression cassette, a plasmid, a vector genome, or the like) for replacement of missing or aberrant gene expression, a coding sequence for expression of a heterologous gene product that provides a therapeutic effect, and the like.

The term “polypeptide” is used interchangeably with the terms “polypeptides” and “protein(s),” and refers to a polymer of amino acid residues. Polypeptides include functional protein fragments of essentially any length as well as full length proteins. The term “peptide”, as used herein, will generally refer to a polypeptide chain of 40 or less amino acids. A “peptide therapeutic” is a peptide having an established therapeutic function.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “monoclonal antibody” as used herein is not limited to antibodies produced through hybridoma technology. The term “monoclonal antibody” refers to an antibody that is derived from a single clone, including any eukaryotic, prokaryotic, or phage clone and not the method by which it is produced. Monoclonal antibodies useful in connection with the present disclosure can be prepared using a wide variety of techniques including, but not limited to, the use of hybridoma, recombinant, and phage display technologies or a combination thereof.

The term “transduction”, as used herein, generally refers to the introduction of foreign nucleic acid into a cell using a viral vector and the term “transfection”, as used herein, generally refers to the process of introducing nucleic acid into cells by non-viral methods. However, in some instances throughout the disclosure, which will be readily apparent to the ordinarily skilled artisan, the terms “transduction” and “transfection” may be used interchangeably. In some instances, use of the term transduction may exclude non-viral delivery of nucleic acids. In some instances, use of the term transfection may exclude viral delivery of nucleic acids.

The term “vector copy number” or “VCN” refers to the number of copies of a vector, or portion thereof, introduced into a cell. The average VCN may be determined from a population of cells or from individual cell colonies. Exemplary methods for determining VCN include polymerase chain reaction (PCR) and flow cytometry.

The terms “virus particles”, “virus”, and the like, refer to an infectious viral agent, including, e.g., baculovirus particles, lentivirus particles, adenovirus particles, and the like. Virus and virus particles may be naturally occurring, recombinant, engineered, or synthetic.

A “vector” or “expression vector” is a replicon, such as plasmid, phage, virus, or cosmid, to which another DNA segment, i.e. an “insert”, may be attached so as to bring about the replication of the attached segment in a cell.

As used herein, the term “retrovirus” refers to an RNA virus that reverse transcribes its genomic RNA into a linear double-stranded DNA copy and subsequently covalently integrates its genomic DNA into a host genome. Retroviruses are a common tool for gene delivery. Illustrative retroviruses include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), Spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immunodeficiency virus (BIV); and simian immunodeficiency virus (SIV). In some embodiments, HIV based vector backbones (i.e., HIV cis-acting sequence elements) may be employed.

Retroviral vectors, and more particularly, lentiviral vectors, may be used in practicing the present invention. Accordingly, the term “retrovirus” or “retroviral vector,” as used herein is meant to include “lentivirus” and “lentiviral vectors” respectively.

### Composition and Methods

The present disclosure includes compositions and methods comprising biliary-therapeutic enhancers for the delivery of therapeutic biologics in situations where bile may be present at the targeted delivery location, thereby providing improved therapeutic effects from the biologic.

As described and demonstrated herein, the presence of bile has a disruptive effect on the activity of therapeutic biologics, decreasing, inhibiting, or ablating the therapeutic effect of such biologics in a live subject. Accordingly, when biologics are delivered to a target location in a subject where bile is present, the therapeutic effect may be reduced, e.g., as compared to the activity expected in the absence of bile, or even lost entirely. This detrimental effect of bile on biologics limits the in vivo locations to which biologic therapeutics may be administered while still achieving a desired therapeutic effect.

Numerous biologic therapeutics have been developed for the treatment of liver conditions that have shown promise in in vitro and in preclinical studies but, when administered systemically to human subjects in clinical trials, many of these biologics do not demonstrate clinical efficacy. One alternative approach to improving liver directed therapies and/or increasing clinical efficacy of liver-directed therapeutic biologics is to concentrate the therapeutic at the liver, thus achieving a high local concentration of the therapeutic in and/or around the liver, e.g., as compared to the relatively lower concentration of the biologic systemically and/or at locations other than the liver. As described herein, increased local concentration of relevant biologic therapeutics in the liver may be achieved by delivery of the biologic directly to the biliary tract, allowing for retrograde movement of the biologic to the liver. While this approach provides various advantages, including increased liver concentration, lower dose, more rapid exposure, etc., the presence of bile in the biliary tract can, as described herein, have a detrimental effect on the activity of the delivered biologic.

The present disclosure is based, at least in part, on the discovery that the addition of certain reagents to mixtures containing bile can reduce the detrimental effects of the bile on the activity of a biologic therapeutic that may, at that time or subsequently, come into contact with the bile-containing mixture, or otherwise increase the activity of the biologic when present in an environment where bile is present. Accordingly, such reagents enhance the effect of therapeutics delivered in the presence of bile and are referred to herein as biliary-therapeutic enhancers. The methods and compositions described herein provide unexpectedly superior results following the direct delivery of biologic therapeutics to the biliary tract, for example various advantages including but not limited to increased local concentration in the liver which results in increased therapeutic efficacy.

Described herein are compositions comprising one or more therapeutic biologics and/or one or more biliary-therapeutic enhancers. In certain embodiments, the composition comprises a combination of at least one therapeutic biologic and at least one biliary-therapeutic enhancer. The compositions of the invention may be pharmaceutical compositions and may further comprise one or more pharmaceutically acceptable excipients, buffers, and/or other reagents. Additional agents may also be included in a separate composition and/or with a composition comprising the one or more therapeutic biologics and/or biliary-therapeutic enhancers. For example, in some instances, a bile acid secretion inhibitor may also be included in a separate composition, and/or with a composition comprising the one or more therapeutic biologics and/or biliary-therapeutic enhancers. Bile acid secretion inhibitors include but are not limited to agonists of the bile acid receptor (BAR), also known as farnesoid X receptor (FXR) or NR1H4 (nuclear receptor subfamily 1, group H, member 4). In certain embodiments, the bile acid secretion inhibitor is an FXR agonist. Any amount (dosage) of therapeutic biologic and biliary therapeutic enhancer may be used in the separate or combined compositions described herein. Such dosages may be readily determined.

Aspects of the instant disclosure include pharmaceutical compositions for performing one or more of the methods described herein where such a pharmaceutical composition may include a therapeutic biologic and/or a biliary-therapeutic enhancer appropriately formulated for administration as described herein. In some instances, a pharmaceutical composition may include a therapeutic biologic and two or more biliary-therapeutic enhancers (e.g., a bile acid sequestrant and a cationic or nonionic amphiphilic transduction enhancer) appropriately formulated for administration as described herein. In some instances, a pharmaceutical composition may include two or more therapeutic biologics and a biliary-therapeutic enhancer appropriately formulated for administration as described herein.

The active agents of the pharmaceutical compositions may be combined, i.e., as a composition of two or more active agents, or may be formulated individually into separate compositions. In some instances, pharmaceutical compositions individually formulated with each active agent may be provided in the form of a kit, as described herein, for treating a subject with a combination treatment of two or more compositions each having one or more of the active agents (e.g., biologic therapeutic, biliary-therapeutic enhancer, bile acid sequestrant, cationic or nonionic amphiphilic transduction enhancer, etc.).

A pharmaceutical composition comprising one or more compounds may be administered to a patient alone, or in combination with other supplementary active agents. The pharmaceutical compositions may be manufactured using any of a variety of processes, including, without limitation, conventional mixing, dissolving, granulating, dragee-making, levigating, emulsifying, encapsulating, entrapping, and lyophilizing Pharmaceutical compositions can take any of a variety of forms including, without limitation, a sterile solution, suspension, emulsion, lyophilisate, tablet, pill, pellet, capsule, powder, syrup, elixir or any other dosage form suitable for administration.

In some embodiments, a pharmaceutical composition may be configured as a liquid preparation or liquid suspension allowing for delivery, including e.g., by injection or infusion, into the biliary tract. Suitable liquid preparations may, depending on the desired formulation, be prepared immediately in advance of use, or delivery, or may be prepared well in advance and stored before use.

A composition described herein comprising one or more compounds may be administered to the host using any convenient means capable of resulting in the desired reduction in disease condition or symptom. Thus, a compound can be incorporated into a variety of formulations for therapeutic administration. More particularly, a compound can be formulated into pharmaceutical compositions by combination with appropriate pharmaceutically acceptable carriers or diluents, and may be formulated into various preparations.

Guidance for the formulation of pharmaceutical compositions is readily available. For example, Remington's Pharmaceutical Sciences, by E. W. Martin, Mack Publishing Co., Easton, Pa., 19th Edition, 1995, describes exemplary formulations (and components thereof) suitable for pharmaceutical delivery of disclosed compounds. Pharmaceutical compositions comprising at least one of the compounds can be formulated for use in human or veterinary medicine. Particular formulations of a disclosed pharmaceutical composition may depend, for example, on the mode of administration and/or on the location of the affected area to be treated. In some embodiments, formulations include a pharmaceutically acceptable carrier in addition to at least one active ingredient. In other embodiments, other medicinal or pharmaceutical agents, for example, with similar, related or complementary effects on the affliction being treated can also be included as active ingredients in a pharmaceutical composition.

Pharmaceutically acceptable carriers useful for the disclosed methods and compositions are readily available. The nature of a pharmaceutical carrier will depend on the particular mode of administration being employed. For example, liquid formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional nontoxic solid carriers can include, for example, pharmaceutical grades of mannitol, lactose, starch, or magnesium stearate. In addition to biologically neutral carriers, pharmaceutical compositions to be administered can optionally contain minor amounts of non-toxic auxiliary substances (e.g., excipients), such as wetting or emulsifying agents, preservatives, and pH buffering agents and the like; for example, sodium acetate or sorbitan monolaurate. Other non-limiting excipients include, nonionic solubilizers, such as cremophor, or proteins, such as human serum albumin or plasma preparations.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, and mannitol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The disclosed pharmaceutical compositions, and/or components thereof, may be formulated as a pharmaceutically acceptable salt of a disclosed compound. Pharmaceutically acceptable salts are non-toxic salts of a free base form of a compound that possesses the desired pharmacological activity of the free base. These salts may be derived from inorganic or organic acids. Non-limiting examples of suitable inorganic acids are hydrochloric acid, nitric acid, hydrobromic acid, sulfuric acid, hydroiodic acid, and phosphoric acid. Non-limiting examples of suitable organic acids are acetic acid, propionic acid, glycolic acid, lactic acid, pyruvic acid, malonic acid, succinic acid, malic acid, maleic acid, fumaric acid, tartaric acid, citric acid, benzoic acid, cinnamic acid, mandelic acid, methanesulfonic acid, ethanesulfonic acid, p-toluenesulfonic acid, methyl sulfonic acid, salicylic acid, formic acid, trichloroacetic acid, trifluoroacetic acid, gluconic acid, asparagic acid, aspartic acid, benzenesulfonic acid, p-toluenesulfonic acid, naphthalenesulfonic acid, and the like. Lists of other suitable pharmaceutically acceptable salts are found in Remington's Pharmaceutical Sciences, 17th Edition, Mack Publishing Company, Easton, Pa., 1985. A pharmaceutically acceptable salt may also serve to adjust the osmotic pressure of the composition.

A pharmaceutical composition comprising one or more component compounds can be formulated into preparations for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, the compositions described herein can be delivered by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are readily available.

The term “unit dosage form,” as used herein, refers to physically discrete units suitable as unitary dosages for human and animal subjects, each unit containing a predetermined quantity of the compositions as described herein (or predetermined quantities thereof) calculated in an amount(s) sufficient to produce the desired effect in association with a pharmaceutically acceptable diluent, carrier or vehicle. The specifications for the compositions described herein depend on the particular compound employed and the effect to be achieved, and the pharmacodynamics associated with each compound in the host. The dosage form of a disclosed pharmaceutical composition will be determined by the mode of administration chosen.

Each therapeutic compound, or individual component, of a composition, can independently be in any dosage form, such as those described herein, and can also be administered in various ways, as described herein. For example, the compounds may be formulated together, in a single dosage unit (that is, combined together in one form such as capsule, tablet, powder, liquid, or suspension, etc.) as a combination product. Alternatively, when not formulated together in a single dosage unit, an individual compound may be administered at the same time as another therapeutic compound or sequentially, in any order thereof. In some instances, for liquid delivery of an amount or dosage provided in solid form (e.g., capsule, tablet, powder, etc.) the solid form may be dissolved, suspended, emulsified, rehydrated, etc., prior to use.

As described in more detail herein, the sequence of delivery of the compositions (steps in the described methods) may vary. For example, without limitation, in some instances the methods may involve contacting a biliary-therapeutic enhancer with bile and subsequently adding or administering a biologic therapeutic. In some embodiments, the biliary-therapeutic enhancer composition may be delivered directly to the biliary tract and the biologic composition may then be subsequently administered, e.g., by the same route directly to the biliary tract. Accordingly, the biliary-therapeutic enhancer and the biologic may be combined in vivo. In some instances, the biliary-therapeutic enhancer and the biologic therapeutic may be contacted with bile simultaneously or at essentially the same time. In some embodiments, the biliary-therapeutic enhancer may be mixed with the biologic prior to administration (ex vivo), prior to delivery directly to the biliary tract. In some instances, a composition that includes both the biliary-therapeutic enhancer and the biologic therapeutic may be contacted together with bile. The compositions described herein, for example the combination compositions, may be prepared at any time prior to administration, including immediately, seconds, minutes, hours, days or months prior to administration. In certain embodiments, composition may be prepared well before administration and the composition may be subsequently administered directly to the biliary tract. Furthermore, sequential and/or concurrent administration of the compositions described herein may be performed in any order and any number of times, including but not limited to one or more sequential and/or administrations of the same or different compositions.

As summarized above, methods of the present disclosure may include contacting a bile-containing solution with a biliary-therapeutic enhancer, a therapeutic biologic and/or one or more compositions as described herein, including for example, where the solution is subsequently or simultaneously contacted with a therapeutic biologic, biliary-therapeutic enhancer, therapeutic biologic and/or one or more compositions. Bile-containing solutions will vary and include essentially any solution of bile or liquid containing bile or primary bile components. Typically, the bile-containing solution comprises bile from the subject to be treated with the compositions or according to the methods described herein. Bile is primarily made up of conjugated bile salts, cholesterol, phospholipid, bilirubin, and electrolytes resulting in a clear, yellow, or orange fluid. Bile is produced by the liver, stored as a concentrate in the gallbladder, and released into the small intestine via the bile ducts when needed for digestion. Bile assists in alkalinizing intestinal contents and in the emulsification, absorption, and digestion of fat. Bile salts, produced in the liver, are made of bile acids that are conjugated with glycine or taurine. Glyco-bile and tauro-bile acids are also referred to as conjugated bile acids. Glycine or taurine bonding increases the water solubility of bile salts. Bile acids are steroid acids derived from cholesterol and may be classified as primary (i.e., those synthesized in the liver, e.g., cholic and chenodeoxycholic acids) and secondary (i.e., those produced from primary bile acids by intestinal bacteria and returned to the liver by enterohepatic circulation, e.g., deoxycholic and lithocholic acids). Human liver synthesizes about 200 to 600 mg of bile acids per day.

Non-limiting examples of bile-containing solutions include bile (e.g., as naturally present in a bile duct; as extracted from a subject; etc.), diluted bile (e.g., as present in a bile duct after addition of or washing with a diluent, buffer, media, or other solution; as prepared from bile ex vivo; etc.), and the like. Bile solutions may vary and may contain various amounts of bile, including but not limited to e.g., 0.01% bile or less, 0.01% bile or more, 0.1% bile or less, 0.1% bile or more, 1% bile or less, 1% bile or more, 2% bile or less, 2% bile or more, 3% bile or less, 3% bile or more, 4% bile or less, 4% bile or more, 5% bile or less, 5% bile or more, 6% bile or less, 6% bile or more, 7% bile or less, 7% bile or more, 8% bile or less, 8% bile or more, 9% bile or less, 9% bile or more, 10% bile or less, 10% bile or more, 15% bile or less, 15% bile or more, 20% bile or less, 20% bile or more, 25% bile or less, 25% bile or more, 30% bile or less, 30% bile or more, 40% bile or less, 40% bile or more, 50% bile or less, 50% bile or more, 60% bile or less, 60% bile or more, 70% bile or less, 70% bile or more, 75% bile or less, 75% bile or more, 80% bile or less, 80% bile or more, 90% bile or less, 90% bile or more, 95% bile or less, 95% bile or more, 97% bile or less, 97% bile or more, 98% bile or less, 98% bile or more, 99% bile or less, 99% bile or more, or 100% bile.

Useful biliary-therapeutic enhancers will vary and may, in some instances, include polymers or resins. In some instances, useful biliary-therapeutic enhancers will include a polyamine polymer or a polyether polymer.

Useful biliary-therapeutic enhancers include but are not limited to e.g., bile acid sequestrants. In some instances, useful bile acid sequestrants may include a polymer and/or resin bile acid sequestrants, such as e.g., a polyamine polymer bile acid sequestrant and/or a polyether polymer bile acid sequestrant.

Bile acid sequestrants include those agents belonging to a class of polymeric resins that bind bile acids in the gastrointestinal tract and prevent their recirculation. Conventionally, medications from this class are administered orally and used to treat high serum cholesterol levels. Bile acid sequestrants are not significantly absorbed from the gut into the bloodstream. Thus, bile acid sequestrants, along with any bile acids bound to the drug, are excreted via the fecal route after passage through the gastrointestinal tract. Non-limiting examples of useful bile acid sequestrants include colesevelam, colestyramine (a.k.a. cholestyramine), colestipol, sevelamer, as well as the various salts and any derivatives thereof. In some instances, a binding-enhancer, such as but not limited to oleic acid, may be used or combined with a one or more subject bile acid sequestrants. Generally, such binding-enhancers increase the bile acid binding capacity of a subject bile acid sequestrant when present together in solution, such as but not limited to e.g., the enhanced binding of sevelamer when present together with oleic acid.

Useful bile acid sequestrants also include those described in U.S. Pat. Nos. 5,607,669, 5,679,717, and 7,229,613, US Patent Publication No. 201000331516, and Camilleri & Gores, Am J Physiol Gastrointerst Liver Physiol (2015) 309(4):G209-G215; the disclosures of which are incorporated herein by reference in their entirety.

Bile acid sequestrants include cross-linked polymers bearing ammonium groups or cationic hydrogels whose sequestration activity is primarily based on the electrostatic interaction between cationically charged polymers and anionically charged bile acids. Suitable bile acid sequestrants include, for example, amphiphilic polymers based on poly(meth)acrylates, poly(meth)acrylamides, polyalkylamines and polyallylamines containing quaternary ammonium groups; cyclodextrin and other saccharide based sequestrants; and sequestrants prepared via molecular imprinting methods, which selectively bind bile acids and salts.

Useful biliary-therapeutic enhancers also include but are not limited to e.g., transduction enhancers. The term “transduction enhancers”, as used herein, generally refers to those agents used to enhance the efficiency of in vitro transduction (or transfection) reactions. As the term includes agents useful in both viral and nonviral introduction of nucleic acids into target cells, useful transduction enhancers include agents that enhance both viral and nonviral transduction and transfection reactions. Transduction enhancers will vary in their mechanism of enhancing transduction and/or transfection. Any amount of the one or more enhancers may be used and such amounts may be readily determined.

Non-limiting examples of transduction enhancers include 16,16-Dimethyl Prostaglandin E2, 1-oleoyl lysophosphatidylcholine, AdenoBlast™ (Nordic Diagnostica AB), AdenoBOOST™ (Sirion-Biotech GmbH), Akti-1/2, BX795, cholesteryl groups tethered oligonucleotides, chondroitan-based proteoglycans, Cyclosporin A, Cyclosporin H, Daunorubicin hydrochloride, DEAE-dextran, Dexamethasone, Eeyarestatin I, Etoposide, F108, F127, glycerol, integrin-binding peptides, LentiBlast™ (Nordic Diagnostica AB), LentiBOOST™ (Sirion-Biotech GmbH), linear dextran nonasaccharide, lysophosphatide, lysophosphatidylcholine, lysophosphatidylethanolamine, lysosome-disruptive peptide, MG 132, PF 03814735, poly(ethylene glycol) (PEG), polybrene, polyethyleneimine (PEI), poly-L-lysine, Prostaglandin E2, protamine sulfate, Protransduzin™ (JPT Peptide Technologies Inc.), Rapamycin, Rosuvastatin calcium, SAHA, Staurosporine, sulfated proteoglycans, TransDux™ MAX (System Biosciences, LLC.), TransPlus™ (ALSTEM, INC.), and the like.

Transduction enhancers useful as biliary-therapeutic enhancers include e.g., cationic or nonionic amphiphilic transduction enhancers. In some embodiments, a cationic transduction enhancer or a combination of multiple different cationic transduction enhancers may be employed. In some embodiments, a nonionic amphiphilic transduction enhancer or a combination of multiple different nonionic amphiphilic transduction enhancers may be employed. In some embodiments, a combination of a cationic transduction enhancer and a nonionic amphiphilic transduction enhancer, a combination of two or more different cationic transduction enhancers and a nonionic amphiphilic transduction enhancer, or a combination of a cationic transduction enhancer and two of more different nonionic amphiphilic transduction enhancers may be employed. In some instances, useful transduction enhancers, such as e.g., cationic or nonionic amphiphilic transduction enhancers, may include a polymer transduction enhancer, such as e.g., a cationic polymer or nonionic polymer amphiphilic transduction enhancer, such as e.g., a polyamine polymer transduction enhancer and/or a polyether polymer transduction enhancer. In some instances, a useful cationic or nonionic amphiphilic transduction enhancer may be polybrene (1,5-dimethyl-1,5-diaza-undeca-methyl-polymethobromide), protamine sulfate, polyethyleneimine (PEI), Poly(ethylene glycol) (PEG), poly-L-lysine, or F108. In some instances, useful transduction enhancers include those described in U.S. Pat. Nos. 9,771,599 and 10,815,498, US Pat. Pub. Nos. 20020019358, 20150064788, 20180016600, and 20200124505, and PCT publications WO2020197400 and WO2018208960; the disclosures of which are incorporated by reference herein in their entirety.

In some instances, useful transduction enhancers are poloxamers, including but not limited to e.g., poloxamers having a molecular weight of 12.8 kDa to about 15 kDa. The term “poloxamer” will be readily understood and refers to a non-ionic triblock copolymer composed of a central hydrophobic chain of polyoxypropylene flanked by two hydrophilic chains of polyoxyethylene. Suitable poloxamer transduction enhancers include triblock copolymers (Pluronics) that contain poly(ethylene glycol)-block-poly(propylene glycol)-block-poly(ethylene glycol) in various ratios. An example of a poloxamer transduction enhancer is Pluronic® F108 or Synperonic® F108 (HO—[CH₂CH₂₀]ₓ—[CH₂C₂H₄₀]_{z}—[CH₂CH₂₀]_{y} with _{x+y}=265.45 and _{z}=50.34).

In some embodiments, the bile acid sequestrant includes compounds having formula (I):

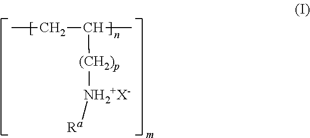

wherein,
p is an integer from 1 to 3,
X is an electrophilic leaving group,
R^{a} is selected from the group consisting of hydrogen, C₁-C₂₀ alkyl, C₂-C₆ alkenyl, C₂-C₆ alkynyl, or C₁-C₂₀ alkylammonium group, and
wherein the alkyl is optionally substituted with OH or alkylammonium.

In some embodiments, for a compound of formula (I), X is selected from C₁ or Br. In some embodiments, X is C₁. In some embodiments, for a compound of formula (I), R^{a} is H or C₁-C₂₀ alkyl. In some embodiments, R^{a} is selected from hydrogen or optionally substituted C₁-C₂₀ alkyl.

In some embodiments, the bile acid sequestrant includes a compound of formula (I), having the structure (Ia):

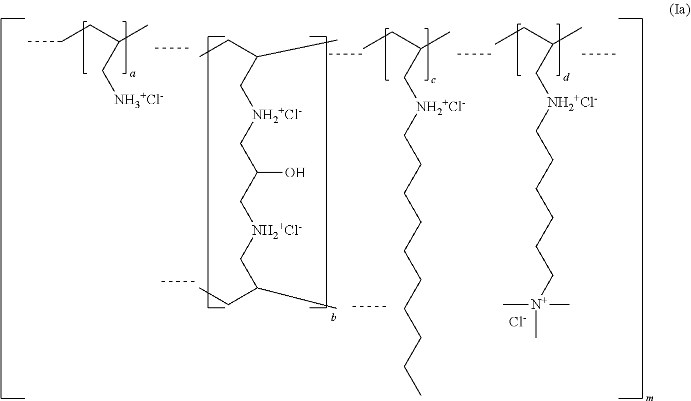

wherein (a) represents allyl amine monomer units that have neither been alkylated by either of the 1-bromodecane or (6-bromohexyl)-trimethylammonium bromide alkylating agents nor cross-linked by epichlorohydrin; (b) represents allyl amine units that have undergone crosslinking with epichlorohydrin; (c) represents allyl amine units that have been alkylated with a decyl group; (d) represents allyl amine units that have been alkylated with a (6-trimethylammonium) hexyl group. In some embodiments, a, b, c and d are each independently present or absent.

In some embodiments, the bile acid sequestrant includes compounds having formula (II):

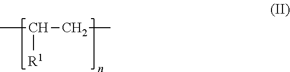

wherein,
R¹ is selected from the group consisting of C₆-C₁₀ aryl or C₂-C₁₀ heteroaryl; wherein the aryl, or heteroaryl, is optionally substituted with 1-3 substituents selected from the group consisting of C₁-C₆ alkyl, C₂-C₆ alkenyl, C₂-C₆ alkynyl, or alkylmmonium halide group.

In some embodiments, for a compound of formula (II), R¹ is C₆-C₁₀ aryl optionally substituted with C₁-C₆ alkyl or alkylmmonium halide group. In some embodiments, R¹ is a benzyl optionally substituted with C₁-C₆ alkyl or alkylmmonium halide group. In some embodiments, R¹ is a benzyl substituted with a C₁-C₃ alkyl group. In some embodiments, R¹ is a benzyl substituted with CH₂N⁺(CH₃)₃Cl— group.

In some embodiments, the bile acid sequestrant includes a compound of formula (II), having the structure (IIa):

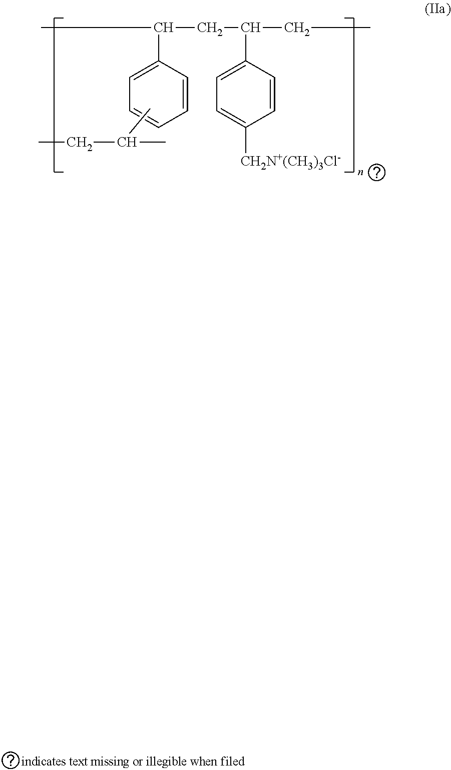

In some embodiments, the biliary-therapeutic enhancer is a cationic or nonionic amphiphilic transduction enhancer and includes compounds having formula (III):

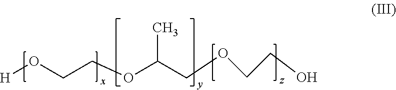

wherein,
each of x, y and z is independently an integer from 1 to 250.

In some embodiments, the bile acid sequestrant includes compounds having the formula (III), wherein x is an integer from 10 to150, y is an integer from 10 to 100 and z is an integer from 10 to 150. In some embodiments, the bile acid sequestrant includes compounds having the formula (III), wherein x and z are each 130-140, and y is 40-60.

In some embodiments, the biliary-therapeutic enhancer is a cationic or nonionic amphiphilic transduction enhancer and includes compounds having formula (IVa) or formula (IVb):

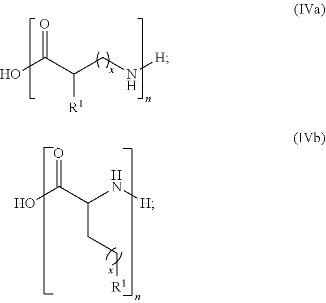

wherein,
x is an integer from 1 to 6, and
R¹ is an amino group, which is optionally substituted with one or more C₁-C₆ alkyl groups.

In some embodiments, the bile acid sequestrants includes compounds having formula (V):

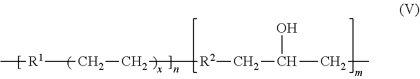

wherein,
each of R¹ and R² is independently selected from the group consisting of amino, alklyamino or alkylmmonium halide group.

In some embodiments, the bile acid sequestrant includes a compound of formula (V), having the structure (Va):

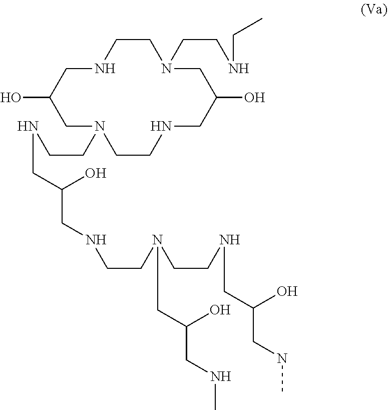

In some embodiments, the biliary-therapeutic enhancer is a cationic or nonionic amphiphilic transduction enhancer and includes compounds having formula (VI):

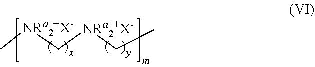

wherein,
x is an integer from 1 to 10,
y is an integer from 1 to 6,
X is an electrophilic leaving group, and
each R^{a} is independently a hydrogen or a C₁-C₆ alkyl group.

In some embodiments, for a compound of formula (I), X is selected from C₁ or Br. In some embodiments, X is Br. In some embodiments, each Ra is hydrogen. In some embodiments, each Ra is C₁-C₃ alkyl. In some embodiments, each Ra is methyl.

In some embodiments, the bile acid sequestrant includes a compound of formula (VI), having the structure (VIa):

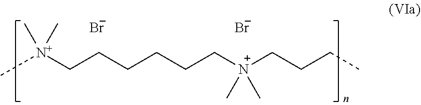

In some embodiments, the bile acid sequestrants includes compounds having formula (VII):

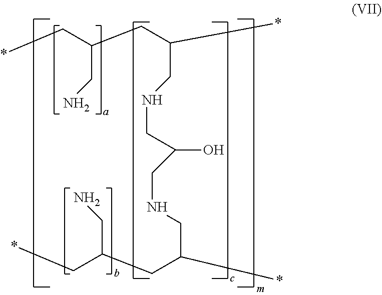

wherein,
a is an integer from 1 to 6,
b is an integer from 1 to 6, and
c is an integer from 1 to 3.

In some embodiments, the bile acid sequestrant includes a compound of formula (VII), wherein a and b represent number of primary amine groups, c represents number of cross-linking groups, and m represents a large number to indicate extended polymer network. In some embodiments, a, b, and c are each independently present or absent. In some embodiments, a+b=9 and c=1.

In all of the above compounds of formulae (I) to (VII), the total number of repeating units can be varied to achieve a desired molecular weight depending on the desired biologic activity. Thus the value of n or m in the compounds of formulae (I) to (VII) can vary from 3 to 10000, 3 to 1000, 3 to 500, 3 to 300 or 3 to 100, or any value in between and encompassing these values. The desired average molecular weight (MW) of each of the compounds of formulae (I), (Ia), (II), (Ila), (III), (IVa), (IVb), (V), (Va), (VI), (VIa), and (VII) can range from about 100 to about 500000 g/mol or Daltons, including from about 100 to about 1000, about 1000 to about 5000, about 5000 to about 10000, about 10000 to about 50000, about 50000 to about 100000, about 100000 to about 300000, about 300000 to about 500000, and ranges between and including any two of these values. In some embodiments, a compound of formula (I) may have m and n value corresponding to a MW of about 500 to about 1000. In some embodiments, a compound of formula (Ia) may have m value corresponding to a MW of about 500 to about 1000. In some embodiments, a compound of formula (II) and (IIa) may have n value corresponding to a MW of about 100 to about 500. In some embodiments, a compound of formula (III) may have x, y and z values corresponding to a MW of about 10000 to about 15000. In some embodiments, a compound of formula (IVa) and (IVb) may have n value corresponding to a MW of about 50000 to about 200000. In some embodiments, a compound of formula (V) may have m and n value corresponding to a MW of about 100 to about 500. In some embodiments, a compound of formula (VI) and (VIa) may have n value corresponding to a MW of about 100 to about 600. In some embodiments, a compound of formula (VII) may have m value corresponding to a MW of about 100 to about 500.

As summarized above, in some embodiments of the herein described methods biliary-therapeutic enhancer may be administered before a therapeutic biologic. Correspondingly, in some embodiments a target cell of the biologic may be contacted with the biliary-therapeutic enhancer before being contacted with the therapeutic biologic. In some embodiments of the herein described methods the biliary-therapeutic enhancer may be administered together, including simultaneously or essentially simultaneously, with the therapeutic biologic. Correspondingly, in some embodiments a target cell of the biologic may be contacted with the biliary-therapeutic enhancer and the therapeutic biologic at the same time or essentially simultaneously. As described herein, in some embodiments, the biliary-therapeutic enhancer and the therapeutic biologic may be co-administered. In some embodiments, the presently disclosed methods may employ co-formulations, including e.g., where the biliary-therapeutic enhancer and the therapeutic biologic are co-formulated.

In some embodiments, two or more biliary-therapeutic enhancers may be employed, including but not limited to e.g., where 2 to 10, 3 to 10, 4 to 10, 5 to 10, 2 to 5, 3 to 5, 2, 2 or more, 3, 3 or more, 4, 4 or more, 5, 5 or more, 6, 6 or more, 7, or 7 or more biliary-therapeutic enhancers are employed. In some embodiments, at least one bile acid sequestrant and at least one transduction enhancer may be employed. In some instances, one bile acid sequestrant and at least two transduction enhancers may be employed. In some instances, two or more bile acid sequestrants may be employed. Where combinations of two or more biliary-therapeutic enhancers are employed, the individual elements of the combinations may be administered and/or contacted with a target cell simultaneously, essentially simultaneously, or sequentially. As such, combinations of two or more biliary-therapeutic enhancers may be co-administered and/or co-formulated.

In some instances, a combination of at least colesevelam and at least polybrene may be employed, including e.g., where colesevelam and polybrene are co-administered, co-formulated, or administered sequentially. In some instances, a combination of at least colesevelam and at least protamine sulfate may be employed, including e.g., where colesevelam and protamine sulfate are co-administered, co-formulated, or administered sequentially. In some instances, a combination of at least colesevelam and at least polyethyleneimine (PEI) may be employed, including e.g., where colesevelam and polyethyleneimine (PEI) are co-administered, co-formulated, or administered sequentially. In some instances, a combination of at least colesevelam and at least poly(ethylene glycol) (PEG) may be employed, including e.g., where colesevelam and poly(ethylene glycol) (PEG) are co-administered, co-formulated, or administered sequentially. In some instances, a combination of at least colesevelam and at least poly-L-lysine may be employed, including e.g., where colesevelam and poly-L-lysine are co-administered, co-formulated, or administered sequentially. In some instances, a combination of at least colesevelam and at least F108 may be employed, including e.g., where colesevelam and F108 are co-administered, co-formulated, or administered sequentially.

In some instances, a combination of at least colestyramine and at least polybrene may be employed, including e.g., where colestyramine and polybrene are co-administered, co-formulated, or administered sequentially. In some instances, a combination of at least colestyramine and at least protamine sulfate may be employed, including e.g., where colestyramine and protamine sulfate are co-administered, co-formulated, or administered sequentially. In some instances, a combination of at least colestyramine and at least polyethyleneimine (PEI) may be employed, including e.g., where colestyramine and polyethyleneimine (PEI) are co-administered, co-formulated, or administered sequentially. In some instances, a combination of at least colestyramine and at least poly(ethylene glycol) (PEG) may be employed, including e.g., where colestyramine and poly(ethylene glycol) (PEG) are co-administered, co-formulated, or administered sequentially. In some instances, a combination of at least colestyramine and at least poly-L-lysine may be employed, including e.g., where colestyramine and poly-L-lysine are co-administered, co-formulated, or administered sequentially. In some instances, a combination of at least colestyramine and at least F108 may be employed, including e.g., where colestyramine and F108 are co-administered, co-formulated, or administered sequentially.

In some instances, a combination of at least colestipol and at least polybrene may be employed, including e.g., where colestipol and polybrene are co-administered, co-formulated, or administered sequentially. In some instances, a combination of at least colestipol and at least protamine sulfate may be employed, including e.g., where colestipol and protamine sulfate are co-administered, co-formulated, or administered sequentially. In some instances, a combination of at least colestipol and at least polyethyleneimine (PEI) may be employed, including e.g., where colestipol and polyethyleneimine (PEI) are co-administered, co-formulated, or administered sequentially. In some instances, a combination of at least colestipol and at least poly(ethylene glycol) (PEG) may be employed, including e.g., where colestipol and poly(ethylene glycol) (PEG) are co-administered, co-formulated, or administered sequentially. In some instances, a combination of at least colestipol and at least poly-L-lysine may be employed, including e.g., where colestipol and poly-L-lysine are co-administered, co-formulated, or administered sequentially. In some instances, a combination of at least colestipol and at least F108 may be employed, including e.g., where colestipol and F108 are co-administered, co-formulated, or administered sequentially.

In some instances, a combination of at least sevelamer and at least polybrene may be employed, including e.g., where sevelamer and polybrene are co-administered, co-formulated, or administered sequentially. In some instances, a combination of at least sevelamer and at least protamine sulfate may be employed, including e.g., where sevelamer and protamine sulfate are co-administered, co-formulated, or administered sequentially. In some instances, a combination of at least sevelamer and at least polyethyleneimine (PEI) may be employed, including e.g., where sevelamer and polyethyleneimine (PEI) are co-administered, co-formulated, or administered sequentially. In some instances, a combination of at least sevelamer and at least poly(ethylene glycol) (PEG) may be employed, including e.g., where sevelamer and poly(ethylene glycol) (PEG) are co-administered, co-formulated, or administered sequentially.

In some instances, a combination of at least sevelamer and at least poly-L-lysine may be employed, including e.g., where sevelamer and poly-L-lysine are co-administered, co-formulated, or administered sequentially. In some instances, a combination of at least sevelamer and at least F108 may be employed, including e.g., where sevelamer and F108 are co-administered, co-formulated, or administered sequentially.

The above-described combinations are merely representative and non-limiting. As provided for the above, this disclosure expressly contemplates and describes all combinations of the herein described biliary-therapeutic enhancers, as well as such combinations with all herein described therapeutic biologics.

Various therapeutic biologics find use in the herein described methods, compositions, and kits. The term “therapeutic biologic” is used interchangeably herein with “biologic therapeutic(s)”, “biologic(s)”, and “therapeutic(s)” and generally refers to agents having a therapeutic effect that are derived from or consist of or contain, at least in part, a polynucleotide or polypeptide, modified or unmodified, and combinations/compositions thereof. Biologics may be isolated from a variety of sources, including but not limited to e.g., human, animal, or microorganism, or may be produced by biotechnology or recombinant methods. Biologics may be wholly or partial synthetic and may include e.g., modified forms of naturally occurring polynucleotides or polypeptides and the like.

In some embodiments, therapeutic biologics employed in the herein described methods may be liable to inactivation by bile. Such bile-labile activity of biologics may be partially or completely inactivated by the presence of bile. The mechanism of inactivation of a particular biologic by bile will vary depending on various factors, including the nature of the biologic, the mechanism of therapeutic action of the biologic, the compositional makeup of the biologic, and the like. For example, in some instances, a bile-labile biologic may be degraded, including partially or completely degraded, in the presence of bile. As a non-exclusive and non-limiting example, biologics that include lipid components, such as but not limited to e.g., enveloped viral vectors, lipid nanoparticles, and the like, may be degraded in the presence of bile, thus rendering such bile-labile biologics inactive. As another non-exclusive and non-limiting example, the activity of a biologic, or some event necessary for the activity of the biologic such as binding to a receptor, ligand, cell, etc., may be blocked or slowed or otherwise inhibited by one or more components of bile such that the biologic is rendered functionally inactive in the presence of bile. As another non-exclusive and non-limiting example, chemical characteristics of a biologic may be altered by the presence of bile, rendering the biologic inactive.

In some embodiments, a therapeutic biologic useful in the herein described methods may be a gene therapy agent. The term “gene therapy”, as used herein, refers to the delivery of nucleic acid therapeutics, sometimes referred to as a genetic payload, into cells of a subject to treat the subject for a condition. Accordingly, a “gene therapy agent”, as used herein, generally refers to the therapeutic nucleic acid and any associated components, if present, used in the delivery of the nucleic acid into the cell, such as delivery vehicles and vectors. Nucleic acids of gene therapy agents will vary and may provide for various functions, including e.g., correction of a defective gene in the host cell, encoding and/or expression of a heterologous gene product in the cell, encoding and/or expression of one or more additional copies of an endogenous gene product in the cell, inhibition of the expression of a gene or a gene product in the cell, or the like. Gene therapy agents include, but not are limited to, viral and/or non-viral vectors carrying one or more polynucleotides encoding one or more proteins (e.g., therapeutic proteins, antibodies and the like). Gene therapy may be administered in vitro to cells within an organism or ex vivo to cells removed from an organism.

Useful nucleic acid therapeutics in gene therapy include but are not limited to e.g., expression cassettes, recombinant mRNA, recombinant vector genomes (such as e.g., recombinant viral genomes), recombinant plasmids, minicircle plasmids, minigenes, microgenes, artificial chromosomes, interfering nucleic acids (e.g., siRNA, shRNA, etc.), and the like. In performing gene therapy nucleic acid therapeutics can be delivered in a variety of ways, including but not limited to e.g., as naked nucleic acid (e.g., naked DNA, naked plasmid, etc.), as a nucleic acid-protein complex (e.g., a DNA-protein complex, an RNA-protein complex such as a ribonucleoprotein (RNP) complex, and the like), as nucleic acid within a viral vector, as nucleic acid within a non-viral vector, and the like. In some instances, proteins may be incorporated into a gene therapy agent, such as but not limited to DNA-binding proteins, RNA-binding proteins, enzymes such as nucleases, and combinations thereof.

Useful gene therapy agents include viral vectors. In some embodiments, useful viral vectors include non-replicating (i.e., replication deficient) viral vectors. Viral vectors may be integrating or non-integrating. Non-limiting examples of useful viral vectors include retroviral vectors, lentiviral vectors, adenoviral vectors, adeno-associated virus (AAV) vectors, and the like. Useful gene therapy agents also include nonviral vectors, which are delivery means that do not employ viral particles and may generally be considered to fall into three categories: naked nucleic acid, particle based (e.g., nanoparticles), or chemical based. Non-limiting examples of nonviral vectors include lipoplexes (e.g., cationic lipid-based lipoplexes), emulsions (such as e.g., lipid nano emulsions), lipid nanoparticles (LNPs), solid lipid nanoparticles, peptide based vectors, polymer based vectors (e.g., polymersomes, polyplexes, polyethylenimine (PEI)-based vectors, chitosan-based vectors, poly (DL-Lactide) (PLA) and poly (DL-Lactide-co-glycoside) (PLGA)-based vectors, dendrimers, vinyl based polymers (e.g., polymethacrylate-based vectors), and the like), inorganic nanoparticles, and the like.

Gene therapy agents (for delivery of nucleic acid therapeutics) may further include promoter sequences (e.g., constitutive, tissue-specific, etc.), terminators, translational regulatory sequences such as ribosome binding sites and internal ribosome entry sites, enhancers, silencers, insulators, boundary elements, replication origins, matrix attachment sites and/or locus control regions. Furthermore, multiple nucleic acid therapeutics can be expressed from one gene therapy agent, for example by linking individual components (transgenes) in one open reading frame separated, for example, by a self-cleaving 2A peptide or IRES sequence.

Vectors, including retroviral vectors, e.g., lentivirus vectors, may include (or exclude as desired where appropriate) various elements, including cis-acting elements, such as promoters, long terminal repeats (LTR), and/or elements thereof, including 5′ LTRs and 3′ LTRs and elements thereof, central polypurine tract (cPPT) elements, DNA flap (FLAP) elements, export elements (e.g., rev response element (RRE), hepatitis B virus post-transcriptional regulatory element (HPRE), etc.), posttranscriptional regulatory elements (e.g., woodchuck hepatitis virus posttranscriptional regulatory element (WPRE), hepatitis B virus regulatory element (HPRE), etc.), polyadenylation sites, transcription termination signals, insulators elements (e.g., β-globin insulator, e.g., chicken HS4), and the like. Other elements that may be present or absent in various vectors include but are not limited to enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, homology regions useful in homology directed repair (HDR), and the like.

Useful LTRs include but are not limited to e.g., those containing U3, R and/or US regions, and portions thereof. LTRs provide functions for the expression of retroviral genes (e.g., promotion, initiation and polyadenylation of gene transcripts) and for viral replication. An LTR can contains numerous regulatory signals including transcriptional control elements, polyadenylation signals and sequences needed for replication and integration of the viral genome. A U3 region may contain enhancer and promoter elements. A US region may contain a polyadenylation sequence. The R (repeat) region is generally flanked by the U3 and US regions. An LTR composed of U3, R and US regions may appear at both the 5′ and 3′ ends of a viral genome. A viral genome may include sequence adjacent to a 5′ LTR that functions in reverse transcription of the genome (e.g., the tRNA primer binding site), for efficient packaging of viral RNA into particles (e.g., the Psi site), and the like.

In various embodiments, vectors comprise modified 5′ LTR and/or 3′ LTRs. Modifications of the 3′ LTR are often made to improve the safety of lentiviral or retroviral systems by rendering viruses replication-defective. As used herein, the term “replication-defective” refers to virus that is not capable of complete, effective replication such that infective virions are not produced (e.g., replication-defective lentiviral progeny). The term “replication-competent” refers to wild-type virus or mutant virus that is capable of replication, such that viral replication of the virus is capable of producing infective virions (e.g., replication-competent lentiviral progeny).

In some embodiments, useful vectors may be self-inactivating. The term “self-inactivating” (SIN) with regards to vectors refers to replication-defective vectors, e.g., retroviral or lentiviral vectors, in which the right (3′) LTR enhancer-promoter region, including e.g., the U3 region, has been modified (e.g., by deletion and/or substitution) to prevent viral transcription beyond the first round of viral replication. In further embodiments, the 3′ LTR may be modified such that the US region is replaced, for example, with a heterologous or synthetic poly(A) sequence, one or more insulator elements, and/or an inducible promoter. It will be readily apparent to the ordinarily skilled artisan where reference to an LTR, e.g., 3′ LTR or 5′ LTR, may include modified LTRs or modifications to LTRs, such as modifications to the 3′ LTR, the LTR, or both 3′ and 5′ LTRs.

In some instances, a retroviral vector may include a heterologous promoter. For example, the U3 region of the 5′ LTR may be replaced with a heterologous promoter to drive transcription of the viral genome during production of viral particles. Examples of useful heterologous promoters include, for example, viral simian virus 40 (SV40) (e.g., early or late), cytomegalovirus (CMV) (e.g., immediate early), Moloney murine leukemia virus (MoMLV), Rous sarcoma virus (RSV), herpes simplex virus (HSV), spleen focus-forming virus (SFFV) promoters and the like. In certain embodiments, the heterologous promoter may be inducible, such that transcription of all or part of the viral genome will occur only when one or more induction factors are present. Induction factors include, but are not limited to, one or more chemical compounds or physiological conditions, e.g., temperature or pH, in which the host cells are cultured.

In some embodiments, viral vectors may comprise a TAR element. The term “TAR” refers to the “trans-activation response” genetic element located in the R region of lentiviral (e.g., HIV) LTRs. This element interacts with the lentiviral trans-activator (tat) genetic element to enhance viral replication. In some embodiments, a vector may not include a TAR element, including e.g., wherein the U3 region of the 5′ LTR is replaced by a heterologous promoter.

In some instances, a vector may be a pseudotyped vector. The terms “pseudotype” or “pseudotyping” as used herein, refer to a virus that has one or more viral envelope proteins that have been substituted with those of another virus possessing preferable characteristics. For example, HIV can be pseudotyped with vesicular stomatitis virus G-protein (VSV-G) envelope proteins. In some embodiments, lentiviral envelope proteins are pseudotyped with VSV-G. In some embodiments, packaging cells which produce recombinant retrovirus, e.g., lentivirus, pseudotyped with the VSV-G envelope glycoprotein may be employed.

Vectors, both viral and nonviral, may include structural and/or genetic elements, or potions thereof, derived from viruses. Retroviral vectors may include structural and/or genetic elements, or potions thereof, derived from retroviruses. Lentiviral vectors may include structural and functional genetic elements, or portions thereof, including LTRs that are primarily derived from a lentivirus. In some instances, hybrid vectors may be employed, including e.g., where a hybrid vector includes an LTR or other nucleic acid containing both retroviral, e.g., lentiviral, sequences and non-retroviral, e.g., non-lentiviral viral, sequences. In some embodiments, a hybrid vector may include a vector comprising retroviral e.g., lentiviral, sequences for reverse transcription, replication, integration and/or packaging.

In some instances, vectors employed in the compositions and methods described herein may include lipid components, such as but not limited to e.g., enveloped viral vectors, lipid nanoparticles, and the like. In some instances, vectors may not have a lipid component. In some instances, the one or more biliary-therapeutic enhancers employed in the compositions and methods described herein may not include any component (or may exclude all components) of a vector, such as e.g., a lipid or polymer present in a nonviral vector, also employed in the method. In some instances, the one or more vectors may not include any component (or may exclude all components) of a biliary-therapeutic enhancer, such as e.g., a transduction enhancer or component thereof present in the enhancer, also employed in the method.

Any vector used in the compositions and methods described herein may be recombinant containing one or more heterologous coding sequences. For example, in some instances, a vector may include a coding sequence for a heterologous protein or peptide useful in treating a condition in the subject. In some instances, the heterologous protein or peptide encoded by the vector may be a liver-directed therapeutic, such as a liver transcription factor, a ligand of a receptor expressed on liver cells, an antibody (such as a monoclonal antibody) that specifically binds an epitope of a protein expressed by liver cells, an enzyme active on liver cells or within the liver microenvironment, or the like. In some instances, the vector may include a therapeutic nucleic acid that inhibits expression of a liver-expressed gene, such as but not limited to e.g., an interfering nucleic acid that inhibits the expression of a gene product of a liver cell. In some instances, liver cells may be employed to express a heterologous gene product, by way of a delivered vector, useful in treating a condition other than a liver condition. As a non-limiting example, a vector encoding a hormone, such as e.g., insulin, may be delivered to liver cells such that the hormone is expressed and secreted by the liver cells to treat a non-hepatic condition, such as e.g., diabetes.

Various payloads may be delivered by gene therapy agents, including but not limited to e.g., noncoding nucleic acids and nucleic acids coding for one or more proteins and/or peptides, including but not limited to e.g., one or more of the therapeutic proteins and/or peptides described herein. In some embodiments, a payload may include nucleic acid sequence coding for an enzyme, such as e.g., a nuclease, a DNA base editor, an RNA editor, or the like. In some embodiments, a payload may include, alone or with other payload elements, a noncoding nucleic acid such as e.g., a microRNA (i.e., miRNA), shRNA, siRNA, piRNA, snoRNA, snRNA, exRNA, scaRNA, lncRNA, guide RNA (gRNA, sgRNA, etc.), or the like.

In some embodiments, a payload of a gene therapy agent may include elements for editing of a target locus. For example, a gene therapy vector may include an exogenous template nucleic acid that includes regions of homology to a target site flanking a sequence that contains the desired edit. Gene editing payload may repair or otherwise introduce a desired edit at a target locus by various mechanism, including e.g., homology directed repair (HDR). Accordingly, in some instances, a gene therapy agent payload may not include a coding sequence, such as e.g., a coding sequence encoding a transgene, therapeutic polypeptide or peptide, or the like. Correspondingly, in some instances, a gene therapy agent payload may exclude one or more elements for expression, including but not limited to e.g., those described herein (e.g., promoters, terminators, enhancers, polyadenylation sequence, etc.). In some instances, a gene therapy agent does not express a protein or polypeptide from a coding sequence and/or does not express a noncoding nucleic acid.

Homology regions targeted to a genomic locus, sometimes referred to as “homology arms” or separately as a “5′ homology arm” and a “3′ homology arm”, share homology to endogenous nucleic acid 5′ and 3′, respectively, of the target site. Homology arms may vary and may range in size from 200 nt or less to 2000 nt or more, including but not limited to e.g., 200 nt or more, 500 nt or more, 500 nt to 1000 nt, etc. Essentially any nucleic acid edit may be introduced and useful edits may include, a single nucleotide edit (i.e., a change of one base for another, e.g., an A to C, an A to T, an A to G, a C to A, a C to G, a C to T, a G to A, a G to C, a G to T, a T to A, a T to C, or a T to G base change), a change of two or more nucleotides (i.e., a change of a base for another at two or more sites), a single nucleotide insertion, an insertion of two or more nucleotides, a single codon insertion (i.e., an insertion of three nucleotides), an insertion of two or more codons, an insertion of a heterologous coding sequence, a single nucleotide deletion, a deletion of two or more nucleotides, a deletion of one or more codons, a deletion of one or more exons, a deletion of a coding region or a portion thereof, a deletion of a noncoding region or a portion thereof, a gene replacement, a replacements of a portion of a gene, etc.

Gene therapy agents containing gene editing payloads may include a variety of other elements, including but not limited to e.g., one or more guide RNAs (e.g., gRNA, sgRNA, etc.), nucleases (e.g., Cas nucleases (e.g., Cas9), zinc finger nucleases (ZFN), transcription activator-like effector nucleases (TALENs), meganucleases, etc.) or sequence encoding one or more nuclease, nickases or sequence encoding one or more nickase, base-editing enzymes (e.g., cytosine base editors, adenine base editors, dual-deaminase editors, etc.) or sequence encoding one or more base-editing enzymes, and the like.

In some embodiments, a therapeutic biologic useful in the herein described methods may be a therapeutic protein or peptide. Useful therapeutic proteins and peptides include, but are not limited to, e.g., secreted factors, hormones, chemokines, cytokines, transcription factors, ligands for receptors, receptor-blocking proteins and peptides, enzymes, extracellular matrix proteins, signaling proteins, antibodies, and the like. In certain embodiments, the therapeutic protein comprises a functional protein lacking (i.e., deficient and/or absent) in a liver disease.

Non-limiting examples of therapeutic proteins and peptides include Lepirudin, Cetuximab, Dornase alfa, Denileukin diftitox, Etanercept, Bivalirudin, Leuprolide, Peginterferon alfa-2a, Alteplase, Interferon alfa-nl, Darbepoetin alfa, Reteplase, Epoetin alfa, Salmon Calcitonin, Interferon alfa-n3, Pegfilgrastim, Sargramostim, Secretin, Peginterferon alfa-2b, Asparaginase, Thyrotropin Alfa, Antihemophilic Factor, Anakinra, Gramicidin D, Intravenous Immunoglobulin, Anistreplase, Insulin Regular, Tenecteplase, Menotropins, Interferon gamma-1b, Interferon Alfa-2a, Recombinant, Coagulation factor VIIa, Oprelvekin, Palifermin, Glucagon recombinant, Aldesleukin, Botulinum Toxin Type B, Omalizumab, Lutropin alfa, Insulin Lispro, Insulin Glargine, Collagenase, Rasburicase, Adalimumab, Imiglucerase, Abciximab, Alpha-1-proteinase inhibitor, Pegaspargase, Interferon beta-1a, Pegademase bovine, Human Serum Albumin, Eptifibatide, Serum albumin iodonated, Infliximab, Follitropin beta, Vasopressin, Interferon beta-1b, Interferon alfacon-1, Hyaluronidase, Insulin, porcine, Trastuzumab, Rituximab, Basiliximab, Muromonab, Digoxin Immune Fab (Ovine), Ibritumomab, Daptomycin, Tositumomab, Pegvisomant, Botulinum Toxin Type A, Pancrelipase, Streptokinase, Alemtuzumab, Alglucerase, Capromab, Laronidase, Urofollitropin, Efalizumab, Serum albumin, Choriogonadotropin alfa, Antithymocyte globulin, Filgrastim, Coagulation factor ix, Becaplermin, Agalsidase beta, Interferon alfa-2b, Oxytocin, Enfuvirtide, Palivizumab, Daclizumab, Bevacizumab, Arcitumomab, Eculizumab, Panitumumab, Ranibizumab, Idursulfase, Alglucosidase alfa, Exenatide, Mecasermin, Pramlintide, Galsulfase, Abatacept, Cosyntropin, Corticotropin, Insulin aspart, Insulin detemir, Insulin glulisine, Pegaptanib, Nesiritide, Thymalfasin, Defibrotide, Natural alpha interferon OR multiferon, Glatiramer acetate, Preotact, Teicoplanin, Canakinumab, Ipilimumab, Sulodexide, Tocilizumab, Teriparatide, Pertuzumab, Rilonacept, Denosumab, Liraglutide, Golimumab, Belatacept, Buserelin, Velaglucerase alfa, Tesamorelin, Brentuximab vedotin, Taliglucerase alfa, Belimumab, Aflibercept, Asparaginase Erwinia chrysanthemi, Ocriplasmin, Glucarpidase, Teduglutide, Raxibacumab, Certolizumab pegol, Insulin, isophane, Epoetin zeta, Obinutuzumab, Fibrinolysin aka plasmin, Follitropin alpha, Romiplostim, Lucinactant, Natalizumab, Aliskiren, Ragweed Pollen Extract, Secukinumab, Somatotropin Recombinant, Drotrecogin alfa, Alefacept, OspA lipoprotein, Urokinase, Abarelix, Sermorelin, Aprotinin, Gemtuzumab ozogamicin, Satumomab Pendetide, Albiglutide, Alirocumab, Ancestim, Antithrombin Alfa, Antithrombin III human, Asfotase Alfa, Atezolizumab, Autologous cultured chondrocytes, Beractant, Blinatumomab, C1 Esterase Inhibitor (Human), Coagulation Factor XIII A-Subunit (Recombinant), Conestat alfa, Daratumumab, Desirudin, Dulaglutide, Elosulfase alfa, Elotuzumab, Evolocumab, Fibrinogen Concentrate (Human), Filgrastim-sndz, Gastric intrinsic factor, Hepatitis B immune globulin, Human calcitonin, Human Clostridium tetani toxoid immune globulin, Human rabies virus immune globulin, Human Rho(D) immune globulin, Hyaluronidase (Human Recombinant), Idarucizumab, Immune Globulin Human, Vedolizumab, Ustekinumab, Turoctocog alfa, Tuberculin Purified Protein Derivative, Simoctocog Alfa, Siltuximab, Sebelipase alfa, Sacrosidase, Ramucirumab, Prothrombin complex concentrate, Poractant alfa, Pembrolizumab, Peginterferon beta-1a, Ofatumumab, Obiltoxaximab, Nivolumab, Necitumumab, Metreleptin, Methoxy polyethylene glycol-epoetin beta, Mepolizumab, Ixekizumab, Insulin Pork, Insulin Degludec, Insulin Beef, Thyroglobulin, Anthrax immune globulin human, Anti-inhibitor coagulant complex, Anti-thymocyte Globulin (Equine), Anti-thymocyte Globulin (Rabbit), Brodalumab, C1 Esterase Inhibitor (Recombinant), Canakinumab, Chorionic Gonadotropin (Human), Chorionic Gonadotropin (Recombinant), Coagulation factor X human, Dinutuximab, Efmoroctocog alfa, Factor IX Complex (Human), Hepatitis A Vaccine, Human Varicella-Zoster Immune Globulin, Ibritumomab tiuxetan, Lenograstim, Pegloticase, Protamine sulfate, Protein S human, Sipuleucel-T, Somatropin recombinant, Susoctocog alfa, Thrombomodulin Alfa, and the like. Methods of the present disclosure may include administering proteins and peptides, such as those identified above, by contacting target cells with the protein or peptide or by contacting target cells with a gene therapy agent encoding the protein or peptide.

In some instances, useful therapeutic proteins include proteins that specifically bind a target protein where such binding results in a therapeutic effect, such as therapeutic antibodies which bind a target protein to produce a therapeutic effect. Non-limiting examples of therapeutic antibodies include 9E10, 8H9, Abagovomab, Abatacept, Abciximab, Abituzumab, Abrilumab, Actoxumab, Adalimumab, Adecatumumab, Aducanumab, Afelimomab, Afutuzumab, Alacizumab pegol, ALD518, Alefacept, Alemtuzumab, Alirocumab, Altumomab pentetate, Amatuximab, Anatumomab mafenatox, Anetumab ravtansine, Anifrolumab, Anrukinzumab, Apolizumab, Arcitumomab, Ascrinvacumab, Aselizumab, Atacicept, Atezolizumab, Atinumab, Atlizumab/tocilizumab, Atorolimumab, AVE1642, Bapineuzumab, Basiliximab, Bavituximab, Bectumomab, Begelomab, Belimumab, Benralizumab, Bertilimumab, Besilesomab, Bevacizumab, Bevacizumab/Ranibizumab, Bezlotoxumab, Biciromab, Bimagrumab, Bimekizumab, Bivatuzumab mertansine, Blinatumomab, Blosozumab, BMS-936559, Bococizumab, Brentuximab, Brentuximabvedotin, Briakinumab, Brodalumab, Brolucizumab, Brontictuzumab, Canakinumab, Cantuzumab mertansine, Cantuzumab ravtansine, Caplacizumab, Capromab pendetide, Carlumab, Catumaxomab, cBR96-doxorubicin immunoconjugate, CDP791, Cedelizumab, Certolizumab, Cetuximab, Ch.14.18, Citatuzumab bogatox, Cixutumumab, Clazakizumab, Clenoliximab, Clivatuzumab tetraxetan, Codrituzumab, Coltuximab ravtansine, Conatumumab, Concizumab, CP-751871, CR6261, Crenezumab, CS-1008, Dacetuzumab, Daclizumab, Dalotuzumab, Dapirolizumab pegol, Daratumumab, Dectrekumab, Demcizumab, Denintuzumab mafodotin, Denosumab, Derlotuximab biotin, Detumomab, Dinutuximab, Diridavumab, Dorlimomab aritox, Drozitumab, Duligotumab, Dupilumab, Durvalumab, Dusigitumab, Ecromeximab, Eculizumab, Edobacomab, Edrecolomab, Efalizumab, Efungumab, Eldelumab, Elgemtumab, Elotuzumab, Elsilimomab, Emactuzumab, Emibetuzumab, Enavatuzumab, Enfortumab vedotin, Enlimomab pegol, Enoblituzumab, Enokizumab, Enoticumab, Ensituximab, Epitumomab cituxetan, Epratuzumab, Erlizumab, Ertumaxomab, Etanercept, Etaracizumab, Etrolizumab, Evinacumab, Evolocumab, Exbivirumab, F19, Fanolesomab, Faralimomab, Farletuzumab, Fasinumab, FBTA05, Felvizumab, Fezakinumab, Ficlatuzumab, Figitumumab, Firivumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gemtuzumab, Gevokizumab, Girentuximab, Glembatumumab vedotin, Golimumab, Gomiliximab, Guselkumab, HGS-ETR2, Ibalizumab, Ibritumomab, Icrucumab, Idarucizumab, Igovomab, IIIA4, IM-2C6, IMAB362, Imalumab, IMC-A12, Imciromab, Imgatuzumab, Inclacumab, Indatuximab ravtansine, Indusatumab vedotin, Infliximab, Inolimomab, Inotuzumab ozogamicin, Intetumumab, Ipilimumab, Iratumumab, Isatuximab, Itolizumab, Ixekizumab, KB004, Keliximab, Labetuzumab, Lambrolizumab, Lampalizumab, Lebrikizumab, Lemalesomab, Lenzilumab, Lerdelimumab, Lexatumumab, Libivirumab, Lifastuzumab vedotin, Ligelizumab, Lilotomab satetraxetan, Lintuzumab, Lirilumab, Lodelcizumab, Lokivetmab, Lorvotuzumab mertansine, Lucatumumab, Lulizumab pegol, Lumiliximab, Lumretuzumab, Mapatumumab, Margetuximab, Maslimomab, Matuzumab, Mavrilimumab, MEDI4736, Mepolizumab, Metelimumab, Milatuzumab, Minretumomab, Mirvetuximab soravtansine, Mitumomab, MK-0646, Mogamulizumab, Morolimumab, Morolimumab immune, Motavizumab, Moxetumomab pasudotox, MPDL33280A, Muromonab-CD3, Nacolomab tafenatox, Namilumab, Naptumomab estafenatox, Narnatumab, Natalizumab, Nebacumab, Necitumumab, Nemolizumab, Nerelimomab, Nesvacumab, Nimotuzumab, Nivolumab, Nofetumomab merpentan, Obiltoxaximab, Obinutuzumab, Ocaratuzumab, Ocrelizumab, Odulimomab, Ofatumumab, Olaratumab, Olokizumab, Omalizumab, Onartuzumab, Ontuxizumab, Opicinumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pasotuxizumab, Pateclizumab, Patritumab, Pembrolizumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, R1507, Racotumomab, Radretumab, Rafivirumab, Ralpancizumab, Ramucirumab, Ranibizumab, Raxibacumab, Refanezumab, Regavirumab, Reslizumab, Rilotumumab, Rinucumab, Rituximab, Robatumumab, Roledumab, Romosozumab, Rontalizumab, Rovelizumab, Ruplizumab, Sacituzumab govitecan, Samalizumab, Sarilumab, Satumomab pendetide, Secukinumab, Seribantumab, Setoxaximab, Sevirumab, SGN-CD19A, SGN-CD33A, Sibrotuzumab, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tarextumab, Tefibazumab, Telimomab aritox, Tenatumomab, Teneliximab, Teplizumab, Teprotumumab, Tesidolumab, Tetulomab, TGN1412, Ticilimumab/tremelimumab, Tigatuzumab, Tildrakizumab, TNX-650, Tocilizumab, Toralizumab, Tosatoxumab, Tovetumab, Tralokinumab, Trastuzumab, TRBS07, Tregalizumab, Tremelimumab, Trevogrumab, Tucotuzumab celmoleukin, Tuvirumab, Ublituximab, Ulocuplumab, Urelumab, Urtoxazumab, Ustekinumab, Vandortuzumab vedotin, Vantictumab, Vanucizumab, Vapaliximab, Varlilumab, Vatelizumab, Vedolizumab, Veltuzumab, Vepalimomab, Vesencumab, Visilizumab, Volociximab, Vorsetuzumab mafodotin, Votumumab, Zalutumumab, Zanolimumab, Zatuximab, Ziralimumab, Zolimomab aritox, and the like. Methods of the present disclosure may include administering antibodies, such as those identified above, by contacting target cells with the antibody or by contacting target cells with a gene therapy agent encoding the antibody.

Any amount (dosage) of therapeutic biologic can be used, which can be readily determined by the skilled artisan. In certain embodiments, the therapeutic biological comprises a viral gene vector, the dosage of which can be readily determined.

In some embodiments, methods of the present disclosure may further include administration of one or more additional agents that inhibit the secretion of bile acids. Such additional agents, referred to herein as bile acid secretion inhibitors, may be administered by essentially any route, including a biliary route as described herein. In some instances, such bile acid secretion inhibitors may be administered before, concurrently, or after administration of a biliary-therapeutic enhancer as described herein. In some instances, such bile acid secretion inhibitors may be administered before, concurrently, or after administration of a biologic therapeutic as described herein. Useful bile acid secretion inhibitors will vary and may include but are not limited to bile acid analogs and derivatives (e.g., chenodeoxycholic acid and derivatives thereof), agonists (e.g., FXR agonists), growth factors, growth factor analogs, and the like. In some instances, useful FXR agonists may include, but are not limited to, obeticholic acid, FGF19 analog, bile acid analogs, or derivatives thereof.

As summarized above, methods of the present disclosure include contacting a biologic therapeutic and a biliary-therapeutic enhancer with bile acid. Accordingly, the methods described herein may include administering the herein described agents to an in vivo location that contains bile, such as e.g., the biliary tract. In some instances, the herein described agents, individually or together as a composition, are administered directly to the biliary tract. In some instances, the herein described agents are administered to the left hepatic duct, the right hepatic duct, the cystic duct, the common hepatic duct, the common bile duct, or some combination thereof, including all of the left hepatic duct, the right hepatic duct, the cystic duct, the common hepatic duct, and the common bile duct.

The compositions and methods described herein may employ retrograde delivery of the therapeutic biologic to the liver through retroductal delivery of the biologic to the biliary tract, including e.g., where retroductal delivery through the left hepatic duct, the right hepatic duct, the common hepatic duct, and/or the common bile duct. In some instances, retrograde delivery to the liver via the biliary tract may involve blocking outflow from the bile duct into the duodenum. When employed, any convenient method of blocking outflow from the bile duct may be employed, including but not limited to e.g., obstructing the ampulla of vater or another point along the biliary tract, e.g., through use of a catheter, such as a balloon catheter, or the like.

Any direct delivery to the biliary tract may be employed in the practice of the invention. For clarity, direct delivery to the biliary tract does not include oral or systemic delivery of any reagent. In some instances, endoscopy may be used for guided delivery of reagents to a target site such as the biliary tract or a site therein. Useful endoscopes may include a camera and one or more additional endoscopic instruments, such as but not limited to e.g., catheters, balloons, probes, stents, and the like. In some embodiments, an endoscope may be guided through a subject's esophagus, stomach and duodenum where, at the ampulla an injecting device is navigated into the common bile duct and agents are directly injected into the biliary tract using the injection device in a retrograde direction towards the liver. In some instances, a component of the endoscopic device may obstruct the ampulla or other portion of the biliary tract, preventing outflow. In some instances, outflow is prevented by inflating a balloon catheter. Optionally, the biliary tract, or a portion thereof, may be flushed prior to injecting agents. Any suitable flushing medium may be employed, including but not limited to e.g., buffered saline.

In some instances, endoscopic retrograde cholangiopancreatography (ERCP), or techniques and/or equipment thereof, for direct delivery of agents to the biliary tract may be employed. In some instances, imaging techniques, such as but not limited to e.g., magnetic resonance cholangiopancreatography (MRCP) and endoscopic ultrasound, may be employed individually, together, and with or without ERCP. In some instances, methods of the present disclosure involve minimally invasive direct delivery of agents to the biliary tract. In some instances, invasive procedures may be employed. In some embodiments, delivery of agents directly to the biliary tract may involve, or be performed as part of, surgical common bile duct exploration. In some instances, a laparotomy may be performed to access the biliary tract or an adjoining organ. In some instances, a catheter may be inserted through the gallbladder and navigated to a desired delivery site. In some instances, a subject may be cannulated for delivery of agents to the biliary tract.

Direct delivery of active therapeutic biologics to the biliary tract, e.g., via retroductal delivery provides numerous advantages. For example, such methods result in increased local concentrations of the therapeutic within the biliary tract and/or within the liver. Accordingly, the increased local concentration of therapeutic provides for an elevated therapeutic effect and/or the ability to deliver less therapeutic while achieving a sufficient therapeutic effect at the targeted location, e.g., the liver, or within targeted cells, e.g., hepatocytes, hepatic stellate cells (HSCs), Kupffer cells (KCs), liver sinusoidal endothelial cells (LSECs), ductal cells, or combinations thereof.

As summarized above, subjects having a variety of conditions, including liver conditions as well as conditions other than those associated with the liver, may be treated according to the methods as described herein. In some instances, a subject may be treated for an inherited condition, such as e.g., a monogenic disease. For example, without limitation, a subject with an inherited condition, such as e.g., a monogenic disease, may be administered a gene therapy targeting the inherited condition through the methods as described herein. In some instances, a subject may be treated for a metabolic disease, including where the metabolic disease may or may not be an inherited condition. Treatment for a metabolic disease, according to the methods as described herein, may include gene therapy and/or delivery of a protein or peptide to treat the subject for the metabolic disease, e.g., through administration of a replacement enzyme or metabolite or the like.

As summarized above, in some instances, subjects treated according to the methods as described herein and/or employing the compositions and/or kits as described herein may be treated for a liver condition. Non-limiting examples of liver conditions that may be treated include acute intermittent porphyria, acute liver failure, alagille syndrome, alcoholic fatty liver disease, alcoholic hepatitis, alcoholic liver cirrhosis, alcoholic liver disease, alpha 1-antitrypsin deficiency, amebic liver abscess, autoimmune hepatitis, biliary liver cirrhosis, budd-chiari syndrome, chemical and drug induced liver injury, cholestasis, chronic hepatitis, chronic hepatitis B, chronic hepatitis C, chronic hepatitis D, end stage liver disease, erythropoietic protoporphyria, fascioliasis, fatty liver disease, focal nodular hyperplasia, hepatic echinococcosis, hepatic encephalopathy, hepatic infarction, hepatic insufficiency, hepatic porphyrias, hepatic tuberculosis, hepatic veno-occlusive disease, hepatitis, hepatocellular carcinoma, hepatoerythropoietic porphyria, hepatolenticular degeneration, hepatomegaly, hepatopulmonary syndrome, hepatorenal syndrome, hereditary coproporphyria, liver abscess, liver cell adenoma, liver cirrhosis, liver failure, liver neoplasm, massive hepatic necrosis, non-alcoholic fatty liver disease, parasitic liver disease, peliosis hepatis, porphyria cutanea tarda, portal hypertension, pyogenic liver abscess, reye syndrome, variegate porphyria, viral hepatitis, viral hepatitis A, viral hepatitis B, viral hepatitis C, viral hepatitis D, viral hepatitis E, and zellweger syndrome, and the like. In some instances, a subject may be treated for fibrosis or a fibrotic condition. In some instances, a subject may be treated for cirrhosis or a cirrhotic condition.

In some instances, e.g., there a bile acid sequestrant is employed as a biliary-therapeutic enhancer, the condition for which a subject is treated according to the methods of the present disclosure may be a condition which is not a condition for which bile acid sequestrants are conventionally administered, including but not limited to e.g., a hyperlipidemia, a secondary dyslipidemia, a bile acid malabsorption condition, or a diabetic condition.

In some instances, the condition for which a subject is treated is not a liver condition. Methods of the present disclosure may be employed to treat various conditions other than liver conditions. For example, the liver, or cells thereof, may be utilized for the expression of a cell extrinsic agent that treats a non-hepatic condition, e.g., in some embodiments an employed biologic may be a gene therapy agent that results in the expression of insulin or another hormone by liver cells to treat a diabetic condition or other endocrine disorder.

The instant methods may include the co-administration of one or more agents. The terms “co-administration” and “in combination with” include the administration of two or more agents either simultaneously, concurrently or sequentially within no specific time limits. In one embodiment, the agents are present in a solution, bodily fluid, target tissue, cell, cellular environment, and/or in a subject's body at the same time or exert their chemical, biological or therapeutic effect(s) at the same time. In one embodiment, the agents are in the same composition or unit dosage form. In other embodiments, the agents are in separate compositions or unit dosage forms. In certain embodiments, a first agent can be administered prior to (e.g., seconds, minutes, 5 minutes, 15 minutes, 30 minutes, 45 minutes, 1 hour, hours, 2 hours, 4 hours, 6 hours, 12 hours, 24 hours, 48 hours, 72 hours, 96 hours, 1 week, weeks, 2 weeks, 3 weeks, 4 weeks, 5 weeks, 6 weeks, 8 weeks, or 12 weeks before), concomitantly with, or subsequent to (e.g., minutes, 5 minutes, 15 minutes, 30 minutes, 45 minutes, 1 hour, hours, 2 hours, 4 hours, 6 hours, 12 hours, 24 hours, 48 hours, 72 hours, 96 hours, 1 week, weeks, 2 weeks, 3 weeks, 4 weeks, 5 weeks, 6 weeks, 8 weeks, or 12 weeks after) the administration of a second agent.

Treatments described herein may be performed chronically (i.e., continuously) or non-chronically (i.e., non-continuously) and may include administration of one or more agents chronically (i.e., continuously) or non-chronically (i.e., non-continuously). Chronic administration of one or more agents according to the methods described herein may be employed in various instances, including e.g., where a subject has a chronic condition, including e.g., a chronic liver condition (e.g., chronic liver disease, cirrhosis, alcoholic liver disease, non-alcoholic fatty liver disease (NAFLD/NASH), chronic viral hepatitis, etc.), a chronic genetic liver condition (alpha-1 antitrypsin deficiency, Hereditary hemochromatosis, Wilson disease, etc.), chronic liver-related autoimmune conditions (e.g., primary biliary cirrhosis (PBC), primary sclerosing cholangitis (PSC), autoimmune hepatitis (AIH), etc.) etc. Administration of one or more agents for a chronic condition may include but is not limited to administration of the agent for multiple months, a year or more, multiple years, etc. Such chronic administration may be performed at any convenient and appropriate dosing schedule including but not limited to e.g., daily, twice daily, weekly, twice weekly, monthly, twice monthly, etc. In some instances, e.g., in the case of correction of a genetic condition or other persistent gene therapies, a chronic condition may be treated by a single or few (e.g., 2, 3, 4, or 5) treatments. Non-chronic administration of one or more agents may include but is not limited to e.g., administration for a month or less, including e.g., a period of weeks, a week, a period of days, a limited number of doses (e.g., less than 10 doses, e.g., 9 doses or less, 8 doses or less, 7 doses or less, etc., including a single dose).

The route of administration may be selected according to a variety of factors including, but not necessarily limited to, the condition to be treated, the formulation and/or device used, the patient to be treated, and the like. Useful routes of administration include but are not limited to oral and parenteral routes, such as intravenous (iv), intraperitoneal (ip), rectal, topical, ophthalmic, nasal, and transdermal. As described herein, pharmaceutical compositions formulated for particular routes of delivery may be employed.

In some embodiments, a biliary or intraductal route of administration may be employed, including where the biliary tract is accessed through any convenient method, including but not limited to surgical laparotomy, ERCP, or the like.

An effective amount of a subject compound will depend, at least, on the particular method of use, the subject being treated, the severity of the affliction, the manner of administration of the therapeutic composition, and the mechanism of action of the therapeutic. A “therapeutically effective amount” of a composition is a quantity of a specified compound sufficient to achieve a desired effect in a subject being treated.

Therapeutically effective doses of a one or more compositions as described herein can be determined by one of skill in the art, with a goal of achieving local (e.g., tissue) concentrations that are at least as high as the EC50 or IC50 of an applicable compound disclosed herein. In the case of gene therapies, depending on the context, a therapeutically effective dose may, in some instances, include transducing or transfecting some desired percentage (e.g., at least 1%, at least 5%, at least 10%, at least 20%, at least 30%, at least 40%, at least 50%, at least 60%, at least 70%, at least 80%, at least 90%, greater than 90%, etc.) of target cells with one or more (e.g., 2 or more, 3 or more, 4 or more, 5 or more, 10 or more, etc.) viral or non-viral particles or copies of exogenous genetic material.

The specific dose level and frequency of dosage for any particular subject may be varied and will depend upon a variety of factors, including the activity of the composition(s), the stability and length of action of that compound, the age, body weight, general health, sex and diet of the subject, mode and time of administration, rate of excretion, drug combination, and severity of the condition of the host undergoing therapy. Non-limiting examples of doses that may be used include doses ranging from 1 ng to 1 gram (or any value therebetween including any nanogram, microgram or gram amount) of one or more of the composition(s) and/or formulations.

The above listed examples of therapies should not be construed as limiting and essentially any appropriate therapy resulting in the desired therapeutic outcome in subjects identified as described may be employed.

### Kits

Aspects of the present disclosure also include kits and, in some instances, devices, for use therewith or therein. The kits may include, e.g., one or more of any of the components described above with respect to the compositions and methods of the present disclosure. Agents may be in separate vessels or may be combined, according to any described or appropriate combination, into shared vessels. Useful vessels include vials, tubes, syringes, bottles, bags, ampules, and the like.

As summarized above, in some instances, the kits of the present disclosure may comprise a composition of the disclosure may be a pharmaceutical composition, including e.g., a pharmaceutical composition that includes an effective amount of a therapeutic biologic and a biliary-therapeutic enhancer. Such a pharmaceutical composition may include a pharmaceutically acceptable carrier configured for delivery to the biliary tract, e.g., as a liquid.

Also provided are kits comprising one or more compositions and/or for use in the methods described herein. The kits include any combination of components and compositions for performing the methods. The kits may include, e.g., one or more of any of the components described above with respect to the methods. Agents may be in separate vessels or may be combined, according to any described or appropriate combination, into shared vessels. Useful vessels include vials, tubes, syringes, bottles, bags, ampules, and the like.

In some embodiments, useful kits may further include a device. Useful devices will vary and may include an injection or infusion device for delivering one or more agents or compositions to a subject. In some instances, kits of the present disclosure may include a delivery device that is a component of, or compatible with a component of, an endoscope for endoscopic delivery of one or more agents or compositions of the present disclosure to a biliary tract of a subject. In some instances, a delivery device of the present disclosure will be compatible with delivery of agents, according to the methods as described herein, during and ERCP procedure.

In addition to the above components, the kits may further include (in certain embodiments) instructions for practicing the methods. These instructions may be present in the kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

Notwithstanding the appended claims, the present disclosure is also defined by the following embodiments:

1. A method of treating a subject for a condition, the method comprising:
administering directly to the biliary tract of the subject an effective amount of a therapeutic biologic and an effective amount of a biliary-therapeutic enhancer thereby treating the subject for the condition.
   2. The method of embodiment 1, wherein the condition is an inherited condition, optionally wherein the inherited condition is a monogenic disease.
   3. The method of embodiment 1 or embodiment 2, wherein the condition is a metabolic disease.
   4. The method of any of the preceding embodiments, wherein the condition is a liver condition.
   5. The method of embodiment 4, wherein the liver condition is selected from the group consisting of: acute intermittent porphyria, acute liver failure, alagille syndrome, alcoholic fatty liver disease, alcoholic hepatitis, alcoholic liver cirrhosis, alcoholic liver disease, alpha 1-antitrypsin deficiency, amebic liver abscess, autoimmune hepatitis, biliary liver cirrhosis, budd-chiari syndrome, chemical and drug induced liver injury, cholestasis, chronic hepatitis, chronic hepatitis b, chronic hepatitis c, chronic hepatitis d, end stage liver disease, erythropoietic protoporphyria, fascioliasis, fatty liver disease, focal nodular hyperplasia, hepatic echinococcosis, hepatic encephalopathy, hepatic infarction, hepatic insufficiency, hepatic porphyrias, hepatic tuberculosis, hepatic veno-occlusive disease, hepatitis, hepatocellular carcinoma, hepatoerythropoietic porphyria, hepatolenticular degeneration, hepatomegaly, hepatopulmonary syndrome, hepatorenal syndrome, hereditary coproporphyria, liver abscess, liver cell adenoma, liver cirrhosis, liver failure, liver neoplasm, massive hepatic necrosis, non-alcoholic fatty liver disease, parasitic liver disease, peliosis hepatis, porphyria cutanea tarda, portal hypertension, pyogenic liver abscess, reye syndrome, variegate porphyria, viral hepatitis, viral hepatitis a, viral hepatitis b, viral hepatitis c, viral hepatitis d, viral hepatitis e, and zellweger syndrome.
   6. The method of any of the preceding embodiments, wherein the administering comprises retroductal delivery of the therapeutic biologic to the liver of the subject thereby resulting in an increase in local hepatic concentration of the therapeutic biologic.
   7. The method of any of the preceding embodiments, wherein the therapeutic biologic is liable to inactivation by bile.
   8. The method of any of the preceding embodiments, wherein the therapeutic biologic comprises a gene therapy agent or a protein.
   9. The method of embodiment 8, wherein the gene therapy agent comprises a nonviral vector or a viral vector.
   10. The method of embodiment 9, wherein the gene therapy agent comprises a lipid nanoparticle or an enveloped viral vector.
   11. The method of embodiment 10, wherein the enveloped viral vector is a lentiviral vector, optionally wherein the lentiviral vector comprises a 5′ LTR, a promoter, a coding sequence, and a 3′ LTR.
   12. The method of embodiment 10, wherein the lipid nanoparticle does not comprise the biliary-therapeutic enhancer.
   13. The method of embodiment 9, wherein the viral vector is an adenovirus vector or an adeno-associated virus (AAV) vector.
   14. The method of embodiment 8, wherein the protein is a therapeutic peptide.
   15. The method of embodiment 8, wherein the protein is an antibody.
   16. The method of any of the preceding embodiments, wherein the biliary-therapeutic enhancer comprises a polyamine or polyether polymer.
   17. The method of any of the preceding embodiments, wherein the biliary-therapeutic enhancer comprises one or more bile acid sequestrants.
   18. The method of embodiment 17, wherein the one or more bile acid sequestrants comprise a compound of formula (I):

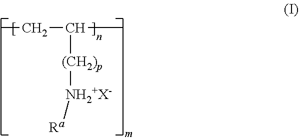

wherein,
   m and n correspond to a MW of about 500 to about 1000,
   p is an integer from 1 to 3,
   X is an electrophilic leaving group,
   R^{a} is selected from the group consisting of hydrogen, C₁-C₂₀ alkyl, C₂-C₆ alkenyl, C₂-C₆ alkynyl, or C₁-C₂₀ alkylammonium group, and
   wherein the alkyl is optionally substituted with OH or alkylammonium.
      19. The method of embodiment 17, wherein the one or more bile acid sequestrants comprise a compound of formula (II):

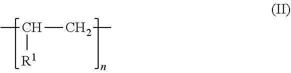

wherein,
   n corresponds to a MW of about 100 to about 500, and
   R¹ is selected from the group consisting of C₆-C₁₀ aryl or C₂-C₁₀ heteroaryl; wherein the aryl, or heteroaryl, is optionally substituted with 1-3 substituents selected from the group consisting of C₁-C₆ alkyl, C₂-C₆ alkenyl, C₂-C₆ alkynyl, or alkylmmonium halide group.
      20. The method of embodiment 17, wherein the one or more bile acid sequestrants comprise a compound of formula (V):

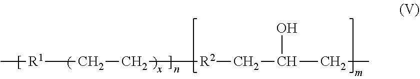

wherein,
   m and n correspond to a MW of about 100 to about 500, and
   each of R¹ and R² is independently selected from the group consisting of amino, alklyamino or alkylmmonium halide group.
      21. The method of embodiment 17, wherein the one or more bile acid sequestrants comprise a compound of formula (VII):

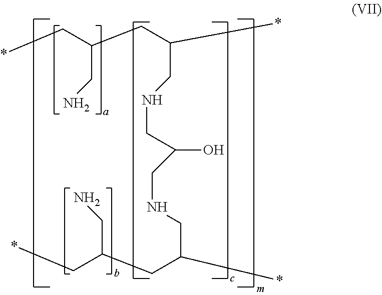

wherein,
   m corresponds to a MW of about 100 to about 500,
   a is an integer from 1 to 6,
   b is an integer from 1 to 6, and
   c is an integer from 1 to 3.
      22. The method of any of embodiments 17 to 21, wherein the one or more bile acid sequestrants are selected from the group consisting of colesevelam, colestyramine, colestipol, and sevelamer.
      23. The method of any of embodiments 1 to 22, wherein the biliary-therapeutic enhancer comprises a cationic or nonionic amphiphilic transduction enhancer.
      24. The method of embodiment 23, wherein the cationic or nonionic amphiphilic transduction enhancer is a compound of formula (III):

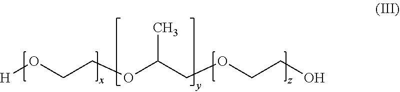

wherein,
   each of x, y and z is independently an integer from 1 to 250.
      25. The method of embodiment 23, wherein the cationic or nonionic amphiphilic transduction enhancer is a compound of formula (IVa) or (IVb):

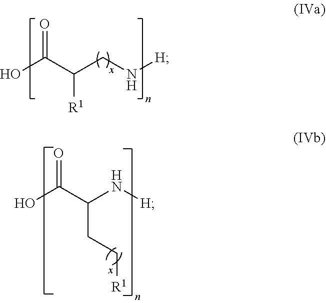

wherein,
   n corresponds to a MW of about 50000 to about 200000,
   x is an integer from 1 to 6, and
   R¹ is an amino group, which is optionally substituted with one or more C₁-C₆ alkyl groups.
      26. The method of embodiment 23, wherein the cationic or nonionic amphiphilic transduction enhancer is a compound of formula (VI):

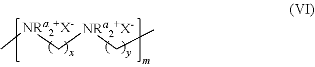

wherein,
   m corresponds to a MW of about 100 to about 600,
   x is an integer from 1 to 10,
   y is an integer from 1 to 6,
   X is an electrophilic leaving group, and
   each R^{a} is independently a hydrogen or a C₁-C₆ alkyl group.
      27. The method of any of embodiments 23 to 26, wherein the cationic or nonionic amphiphilic transduction enhancer is selected from the group consisting of polybrene, protamine sulfate, polyethyleneimine (PEI), Poly(ethylene glycol) (PEG), poly-L-lysine, and F108.
      28. The method of any of the preceding embodiments, wherein the therapeutic biologic and the biliary-therapeutic enhancer are co-administered.
      29. The method of any of the preceding embodiments, wherein the therapeutic biologic and the biliary-therapeutic enhancer are co-formulated in a single pharmaceutical composition.
      30. The method of any of the preceding embodiments, wherein the biliary-therapeutic enhancer is administered before the therapeutic biologic.
      31. The method of any of the preceding embodiments, wherein the method comprises administering two or more biliary-therapeutic enhancers.
      32. The method of embodiment 31, wherein the two or more biliary-therapeutic enhancers comprise a bile acid sequestrant and a cationic or nonionic amphiphilic transduction enhancer.
      33. The method of embodiment 31 or 32, wherein the two or more biliary-therapeutic enhancers are co-administered.
      34. The method of embodiment 33, wherein the two or more biliary-therapeutic enhancers are co-formulated in a single pharmaceutical composition.
      35. The method of any of the preceding embodiments, further comprising administering to the subject a bile acid secretion inhibitor.
      36. The method of embodiment 35, wherein the bile acid secretion inhibitor is an FXR agonist, optionally wherein the FXR agonist is selected from obeticholic acid or an FGF19 analog.
      37. The method of any of the preceding embodiments, wherein the condition is not a hyperlipidemia, a secondary dyslipidemia, a bile acid malabsorption condition, or a diabetic condition.
      38. A pharmaceutical composition comprising:
a pharmaceutically acceptable carrier configured as a liquid for delivery to the biliary tract;
an effective amount of a therapeutic biologic; and/or
a biliary-therapeutic enhancer.
   39. The composition of embodiment 38, wherein the therapeutic biologic is liable to inactivation by bile.
   40. The composition of embodiment 38 or 39, wherein the therapeutic biologic comprises a gene therapy agent or a protein.
   41. The composition of embodiment 40, wherein the gene therapy agent comprises a nonviral vector or a viral vector.
   42. The composition of embodiment 41, wherein the gene therapy agent comprises a lipid nanoparticle or an enveloped viral vector.
   43. The composition of embodiment 42, wherein the enveloped viral vector is a lentiviral vector.
   44. The composition of embodiment 42, wherein the lipid nanoparticle does not comprise the biliary-therapeutic enhancer.
   45. The composition of embodiment 41, wherein the viral vector is an adenovirus vector or an adeno-associated virus (AAV) vector.
   46. The composition of embodiment 40, wherein the protein is a therapeutic peptide.
   47. The composition of embodiment 40, wherein the protein is an antibody.
   48. The composition of any of embodiments 38 to 40, wherein the biliary-therapeutic enhancer comprises a polyamine or polyether polymer.
   49. The composition of any of embodiments 38 to 48, wherein the biliary-therapeutic enhancer is a bile acid sequestrant.
   50. The composition of embodiment 49, wherein the bile acid sequestrant is a compound of formula (I):

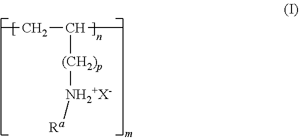

wherein,
   m and n correspond to a MW of about 500 to about 1000,
   p is an integer from 1 to 3,
   X is an electrophilic leaving group,
   R^{a} is selected from the group consisting of hydrogen, C₁-C₂₀ alkyl, C₂-C₆ alkenyl, C₂-C₆ alkynyl, or C₁-C₂₀ alkylammonium group, and
   wherein the alkyl is optionally substituted with OH or alkylammonium.
      51. The composition of embodiment 49, wherein the bile acid sequestrant is a compound of formula (II):

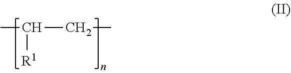

wherein,
   n corresponds to a MW of about 100 to about 500, and
   R¹ is selected from the group consisting of C₆-C₁₀ aryl or C₂-C₁₀ heteroaryl; wherein the aryl, or heteroaryl, is optionally substituted with 1-3 substituents selected from the group consisting of C₁-C₆ alkyl, C₂-C₆ alkenyl, C₂-C₆ alkynyl, or alkylmmonium halide group.
      52. The composition of embodiment 49, wherein the bile acid sequestrant is a compound of formula (V):

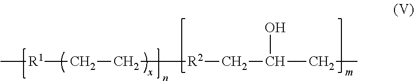

wherein,
   m and n correspond to a MW of about 100 to about 500, and
   each of R¹ and R² is independently selected from the group consisting of amino, alklyamino or alkylmmonium halide group.
      53. The composition of embodiment 49, wherein the bile acid sequestrant is a compound of formula (VII):

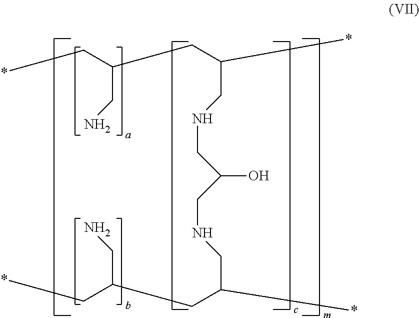

wherein,
   m corresponds to a MW of about 100 to about 500,
   a is an integer from 1 to 6,
   b is an integer from 1 to 6, and
   c is an integer from 1 to 3.
      54. The composition of any of embodiments 49 to 53, wherein the bile acid sequestrant is selected from the group consisting of colesevelam, colestyramine, colestipol, and sevelamer.
      55. The composition of embodiments 49 to 54, wherein the bile acid sequestrant is present in the pharmaceutical composition in a sub-therapeutic amount.
      56. The composition of any of embodiments 38 to 48, wherein the biliary-therapeutic enhancer is a cationic or nonionic amphiphilic transduction enhancer.
      57. The composition of embodiment 56, wherein the cationic or nonionic amphiphilic transduction enhancer is a compound of formula (III):

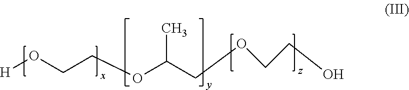

wherein,
   each of x, y and z is independently an integer from 1 to 250.
      58. The composition of embodiment 56, wherein the cationic or nonionic amphiphilic transduction enhancer is a compound of formula (IVa) or (IVb):

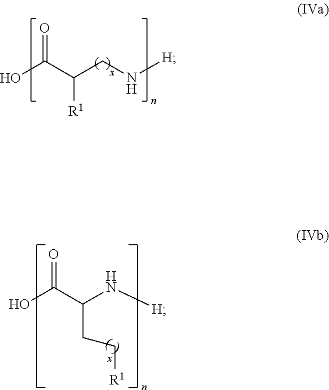

wherein,
   n corresponds to a MW of about 50000 to about 200000,
   x is an integer from 1 to 6, and
   R¹ is an amino group, which is optionally substituted with one or more C₁-C₆ alkyl groups.
      59. The composition of embodiment 56, wherein the cationic or nonionic amphiphilic transduction enhancer is a compound of formula (VI):

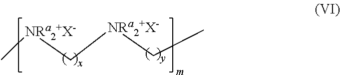

wherein,
   m corresponds to a MW of about 100 to about 600,
   x is an integer from 1 to 10,
   y is an integer from 1 to 6,
   X is an electrophilic leaving group, and
   each R^{a} is independently a hydrogen or a C₁-C₆ alkyl group.
      60. The composition of any of embodiments 56 to 59, wherein the cationic or nonionic amphiphilic transduction enhancer is selected from the group consisting of polybrene, protamine sulfate, polyethyleneimine (PEI), Poly(ethylene glycol) (PEG), poly-L-lysine, and F108.
      61. A method of treating a subject for a condition, the method comprising:
administering directly to the biliary tract of the subject an effective amount of the pharmaceutical composition of any of embodiments 38 to 60, thereby treating the subject for the condition.
62. The method of embodiment 61, wherein the condition is an inherited condition, optionally wherein the inherited condition is a monogenic disease.
   63. The method of embodiment 61 or embodiment 62, wherein the condition is a metabolic disease.
   64. The method of any of embodiments 61 to 63, wherein the condition is a liver condition.
   65. The method of embodiment 64, wherein the liver condition is selected from the group consisting of: acute intermittent porphyria, acute liver failure, alagille syndrome, alcoholic fatty liver disease, alcoholic hepatitis, alcoholic liver cirrhosis, alcoholic liver disease, alpha 1-antitrypsin deficiency, amebic liver abscess, autoimmune hepatitis, biliary liver cirrhosis, budd-chiari syndrome, chemical and drug induced liver injury, cholestasis, chronic hepatitis, chronic hepatitis b, chronic hepatitis c, chronic hepatitis d, end stage liver disease, erythropoietic protoporphyria, fascioliasis, fatty liver disease, focal nodular hyperplasia, hepatic echinococcosis, hepatic encephalopathy, hepatic infarction, hepatic insufficiency, hepatic porphyrias, hepatic tuberculosis, hepatic veno-occlusive disease, hepatitis, hepatocellular carcinoma, hepatoerythropoietic porphyria, hepatolenticular degeneration, hepatomegaly, hepatopulmonary syndrome, hepatorenal syndrome, hereditary coproporphyria, liver abscess, liver cell adenoma, liver cirrhosis, liver failure, liver neoplasm, massive hepatic necrosis, non-alcoholic fatty liver disease, parasitic liver disease, peliosis hepatis, porphyria cutanea tarda, portal hypertension, pyogenic liver abscess, reye syndrome, variegate porphyria, viral hepatitis, viral hepatitis a, viral hepatitis b, viral hepatitis c, viral hepatitis d, viral hepatitis e, and zellweger syndrome.
   66. The method of any of embodiments 61 to 65, wherein the administering comprises retroductal delivery of the therapeutic biologic to the liver of the subject thereby resulting in an enhanced local hepatic concentration of the therapeutic biologic.
   67. A kit comprising:
a liquid pharmaceutically acceptable carrier;
a therapeutic biologic; and
a biliary-therapeutic enhancer.
   68. The kit according to embodiment 67, wherein the biliary-therapeutic enhancer and the liquid pharmaceutically acceptable carrier are formulated together in a vessel.
   69. The kit of embodiments 67 or 68, wherein the therapeutic biologic is liable to inactivation by bile.
   70. The kit of any of embodiments 67 to 69, wherein the therapeutic biologic comprises a gene therapy agent or a protein.
   71. The kit of embodiment 70, wherein the gene therapy agent comprises a nonviral vector or a viral vector.
   72. The kit of embodiment 71, wherein the gene therapy agent comprises a lipid nanoparticle or an enveloped viral vector.
   73. The kit of embodiment 72, wherein the enveloped viral vector is a lentiviral vector.
   74. The kit of embodiment 72, wherein the lipid nanoparticle does not comprise the biliary-therapeutic enhancer.
   75. The kit of embodiment 71, wherein the viral vector is an adenovirus vector or an adeno-associated virus (AAV) vector.
   76. The kit of embodiment 70, wherein the protein is a therapeutic peptide.
   77. The kit of embodiment 70, wherein the protein is an antibody.
   78. The kit of any of embodiments 67 to 77, wherein the biliary-therapeutic enhancer comprises a polyamine or polyether polymer.
   79. The kit of any of embodiments 67 to 78, wherein the biliary-therapeutic enhancer is a bile acid sequestrant.
   80. The kit of embodiment 79, wherein the bile acid sequestrant is a compound of formula (I):

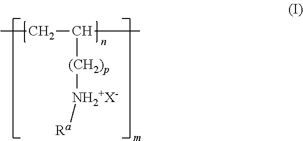

wherein,
   m and n correspond to a MW of about 500 to about 1000,
   p is an integer from 1 to 3,
   X is an electrophilic leaving group,
   R^{a} is selected from the group consisting of hydrogen, C₁-C₂₀ alkyl, C₂-C₆ alkenyl, C₂-C₆ alkynyl, or C₁-C₂₀ alkylammonium group, and
   wherein the alkyl is optionally substituted with OH or alkylammonium.
      81. The kit of embodiment 79, wherein the bile acid sequestrant is a compound of formula (II):

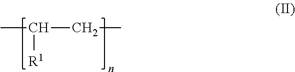

wherein,
   n corresponds to a MW of about 100 to about 500, and
   R¹ is selected from the group consisting of C₆-C₁₀ aryl or C₂-C₁₀ heteroaryl; wherein the aryl, or heteroaryl, is optionally substituted with 1-3 substituents selected from the group consisting of C₁-C₆ alkyl, C₂-C₆ alkenyl, C₂-C₆ alkynyl, or alkylmmonium halide group.
      82. The kit of embodiment 79, wherein the bile acid sequestrant is a compound of formula (V):

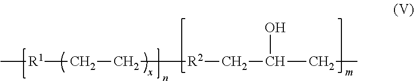

wherein,
   m and n correspond to a MW of about 100 to about 500, and
   each of R¹ and R² is independently selected from the group consisting of amino, alklyamino or alkylmmonium halide group.
      83. The kit of embodiment 79, wherein the bile acid sequestrant is a compound of formula (VII):

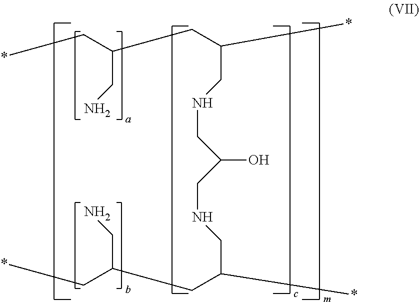

wherein,
   m corresponds to a MW of about 100 to about 500,
   a is an integer from 1 to 6,
   b is an integer from 1 to 6, and
   c is an integer from 1 to 3.
      84. The kit of any of embodiments 79 to 83, wherein the bile acid sequestrant is selected from the group consisting of colesevelam, colestyramine, colestipol, and sevelamer.
      85. The kit of any of embodiments 67 to 78, wherein the biliary-therapeutic enhancer is a cationic or nonionic amphiphilic transduction enhancer.
      86. The composition of embodiment 85, wherein the cationic or nonionic amphiphilic transduction enhancer is a compound of formula (III):

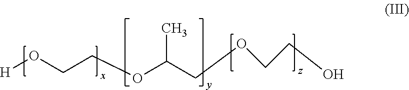

wherein,
   each of x, y and z is independently an integer from 1 to 250.
      87. The composition of embodiment 85, wherein the cationic or nonionic amphiphilic transduction enhancer is a compound of formula (IVa) or (IVb):

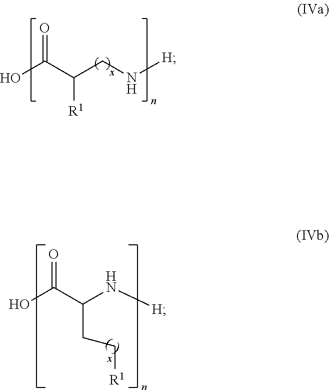

wherein,
   n corresponds to a MW of about 50000 to about 200000,
   x is an integer from 1 to 6, and
   R¹ is an amino group, which is optionally substituted with one or more C₁-C₆ alkyl groups.
      88. The composition of embodiment 85, wherein the cationic or nonionic amphiphilic transduction enhancer is a compound of formula (VI):

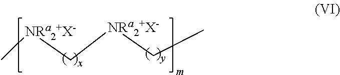

wherein,
   m corresponds to a MW of about 100 to about 600,
   x is an integer from 1 to 10,
   y is an integer from 1 to 6,
   X is an electrophilic leaving group, and
   each R^{a} is independently a hydrogen or a C₁-C₆ alkyl group.
      89. The kit of any of embodiments 85 to 88, wherein the cationic or nonionic amphiphilic transduction enhancer is selected from the group consisting of polybrene, protamine sulfate, polyethyleneimine (PEI), Poly(ethylene glycol) (PEG), poly-L-lysine, and F108.
      90. The kit of any of embodiments 67 to 89, further comprising a device configured for retroductal delivery of the therapeutic biologic to the liver.
      91. Use of one or more compositions and/or one or more kits of any of the preceding embodiments to treat a subject for a condition, optionally wherein the condition is a liver condition, optionally wherein the liver condition is selected from the group consisting of: acute intermittent porphyria, acute liver failure, alagille syndrome, alcoholic fatty liver disease, alcoholic hepatitis, alcoholic liver cirrhosis, alcoholic liver disease, alpha 1-antitrypsin deficiency, amebic liver abscess, autoimmune hepatitis, biliary liver cirrhosis, budd-chiari syndrome, chemical and drug induced liver injury, cholestasis, chronic hepatitis, chronic hepatitis b, chronic hepatitis c, chronic hepatitis d, end stage liver disease, erythropoietic protoporphyria, fascioliasis, fatty liver disease, focal nodular hyperplasia, hepatic echinococcosis, hepatic encephalopathy, hepatic infarction, hepatic insufficiency, hepatic porphyrias, hepatic tuberculosis, hepatic veno-occlusive disease, hepatitis, hepatocellular carcinoma, hepatoerythropoietic porphyria, hepatolenticular degeneration, hepatomegaly, hepatopulmonary syndrome, hepatorenal syndrome, hereditary coproporphyria, liver abscess, liver cell adenoma, liver cirrhosis, liver failure, liver neoplasm, massive hepatic necrosis, non-alcoholic fatty liver disease, parasitic liver disease, peliosis hepatis, porphyria cutanea tarda, portal hypertension, pyogenic liver abscess, reye syndrome, variegate porphyria, viral hepatitis, viral hepatitis a, viral hepatitis b, viral hepatitis c, viral hepatitis d, viral hepatitis e, and zellweger syndrome.
      92. Use of a one or more compositions of any of the preceding embodiments and/or a liquid composition comprising a biliary-therapeutic enhancer for delivery of a therapeutic biologic to the biliary tract of a subject to treat the subject for a liver condition.
      93. The use according to embodiment 92, wherein the biliary-therapeutic enhancer is a bile acid sequestrant, optionally wherein the bile acid sequestrant is a polyamine or polyether polymer.
      94. The use according to embodiment 92, wherein the biliary-therapeutic enhancer is a cationic or nonionic amphiphilic transduction enhancer, optionally wherein the cationic or nonionic amphiphilic transduction enhancer is a polyamine or polyether polymer.
      95. A method of transducing or transfecting a cell, the method comprising:
contacting the cell in the presence of bile with a biliary-transduction enhancer to generate a transduction/transfection composition; and
contacting the transduction/transfection composition with a gene therapy agent comprising an exogenous nucleic acid under conditions sufficient for transduction or transfection of the exogenous nucleic into the cell, thereby transducing or transfecting the cell with the exogenous nucleic acid.
   96. The method of embodiment 95, wherein the cell is a liver cell, optionally wherein the liver cell is a hepatocyte.
   97. The method of embodiment 95 or 96, wherein the gene therapy agent comprises a nonviral vector or a viral vector.
   98. The method of embodiment 97, wherein the gene therapy agent comprises a lipid nanoparticle or an enveloped viral vector.
   99. The method of embodiment 98, wherein the enveloped viral vector is a lentiviral vector.
   100. The method of embodiment 98, wherein the lipid nanoparticle does not comprise the biliary-therapeutic enhancer.
   101. The method of embodiment 97, wherein the viral vector is an adenovirus vector or an adeno-associated virus (AAV) vector.
   102. The method of any of embodiments 95 to 101, wherein the exogenous nucleic acid comprises a coding sequence or portion thereof, optionally wherein the coding sequence encodes a transcription factor, a therapeutic peptide, an antibody or a portion thereof.
   103. The method of any of embodiments 95 to 101, wherein the exogenous nucleic acid comprises a noncoding sequence.
   104. The method of any of embodiments 95 to 103, wherein the biliary-therapeutic enhancer comprises a polyamine or polyether polymer.
   105. The method of any of embodiments 95 to 104, wherein the biliary-therapeutic enhancer comprises one or more bile acid sequestrants.
   106. The method of embodiment 105, wherein the one or more bile acid sequestrants comprise a compound of formula (I):

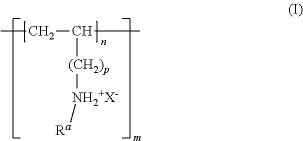

wherein,
   m and n correspond to a MW of about 500 to about 1000,
   p is an integer from 1 to 3,
   X is an electrophilic leaving group,
   R^{a} is selected from the group consisting of hydrogen, C₁-C₂₀ alkyl, C₂-C₆ alkenyl, C₂-C₆ alkynyl, or C₁-C₂₀ alkylammonium group, and
   wherein the alkyl is optionally substituted with OH or alkylammonium.
      107. The method of embodiment 105, wherein the one or more bile acid sequestrants comprise a compound of formula (II):

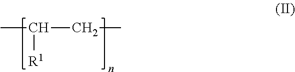

wherein,
   n corresponds to a MW of about 100 to about 500, and
   R¹ is selected from the group consisting of C₆-C₁₀ aryl or C₂-C₁₀ heteroaryl; wherein the aryl, or heteroaryl, is optionally substituted with 1-3 substituents selected from the group consisting of C₁-C₆ alkyl, C₂-C₆ alkenyl, C₂-C₆ alkynyl, or alkylmmonium halide group.
      108. The method of embodiment 105, wherein the one or more bile acid sequestrants comprise a compound of formula (V):

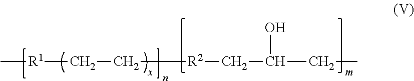

wherein,
   m and n correspond to a MW of about 100 to about 500, and
   each of R¹ and R² is independently selected from the group consisting of amino, alklyamino or alkylmmonium halide group.
      109. The method of embodiment 105, wherein the one or more bile acid sequestrants comprise a compound of formula (VII):

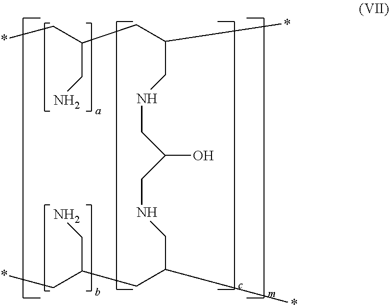

wherein,
   m corresponds to a MW of about 100 to about 500,
   a is an integer from 1 to 6,
   b is an integer from 1 to 6, and
   c is an integer from 1 to 3.
      110. The method of any of embodiments 105 to 109, wherein the one or more bile acid sequestrants are selected from the group consisting of colesevelam, colestyramine, colestipol, and sevelamer.
      111. The method of any of embodiments 95 to 110, wherein the biliary-therapeutic enhancer comprises a cationic or nonionic amphiphilic transduction enhancer.
      112. The method of embodiment 111, wherein the cationic or nonionic amphiphilic transduction enhancer is a compound of formula (III):

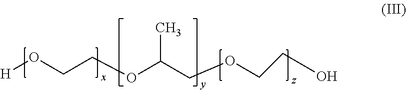

wherein,
   each of x, y and z is independently an integer from 1 to 250.
      113. The method of embodiment 111, wherein the cationic or nonionic amphiphilic transduction enhancer is a compound of formula (IVa) or (IVb):

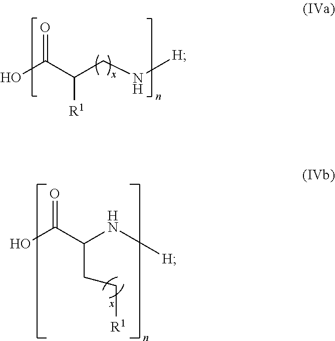

wherein,
   n corresponds to a MW of about 50000 to about 200000,
   x is an integer from 1 to 6, and
   R¹ is an amino group, which is optionally substituted with one or more C₁-C₆ alkyl groups.
      114. The method of embodiment 111, wherein the cationic or nonionic amphiphilic transduction enhancer is a compound of formula (VI):

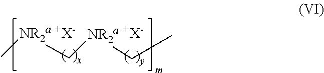

wherein,
   m corresponds to a MW of about 100 to about 600,
   x is an integer from 1 to 10,
   y is an integer from 1 to 6,
   X is an electrophilic leaving group, and
   each R^{a} is independently a hydrogen or a C₁-C₆ alkyl group.
      115. The method of any of embodiments 111 to 114, wherein the cationic or nonionic amphiphilic transduction enhancer is selected from the group consisting of polybrene, protamine sulfate, polyethyleneimine (PEI), Poly(ethylene glycol) (PEG), poly-L-lysine, and F108.
      116. The method of any of embodiments 105 to 115, wherein contacting with the biliary-transduction enhancer comprises contacting the cell with both a bile acid sequestrant and a cationic or nonionic amphiphilic transduction enhancer.
      117. The method of any of embodiments 95 to 116, wherein the biliary-transduction enhancer and the gene therapy agent are present together in a formulation prior to the contacting.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, part are parts by weight, molecular weight is average molecular weight, temperature is in degrees Centigrade, and pressure is at or near atmospheric.

General methods in molecular and cellular biochemistry can be found in such standard textbooks as Molecular Cloning: A Laboratory Manual, 4th Ed. (Sambrook et al., Cold Spring Harbor Laboratory Press 2012); Short Protocols in Molecular Biology, 4th Ed. (Ausubel et al. eds., John Wiley & Sons 1999); Protein Methods (Bollag et ak, John Wiley & Sons 1996); and Cell and Tissue Culture: Laboratory Procedures in Biotechnology (Doyle & Griffiths, John Wiley & Sons 1998), the disclosures of which are incorporated herein by reference. Reagents, antibodies, cells, tissue samples, etc., and kits referred to in this disclosure are available from commercial vendors such, but not limited to, those vendors identified herein.

### Example 1: Effect of Bile on Lentiviral Transduction

An assay was designed to evaluate the effect of the presence of bile on lentiviral transduction of human cells, including human liver cells. A lentiviral vector (LV-SFFV-Luc2-P2A-EmGFP) encoding firefly luciferase (Luc2) and Emerald GFP (EmGFP) reporters, separated by a self-cleaving peptide (P2A), under control of the spleen focus-forming virus (SFFV) promoter was used to facilitate detection of successfully transduced HepG2 or HeLa-RC32 cells in the absence or presence of Sprague Dawley rat bile at various concentrations. Briefly, LV-SFFV-Luc2-P2A-EmGFP vector at 10 MOI was incubated in separate vessels with 100% PBS, 99.9% PBS/0.1% bile, 99% PBS/1% bile, 90% PBS/10% bile, 50% PBS/50% bile, or 100% bile for 1 hour at 37 deg. C. Then cell media, containing cells and transduction enhancer, was mixed with the vector solutions and incubated for 72 hours to allow transduction to take place. To quantitatively assess transduction and lentiviral vector activity after bile exposure the ONE-glo Luciferase Assay System (Promega, Madison, WI) was used. To assay cell viability and the cellular toxicity of bile, cells were incubated in the preceding bile, cell media, and transduction enhancer mixtures, without lentiviral vector, for 72 hours and plates were assayed using the CellTiter-Glo assay system (Promega, Madison, WI).

Reporter was readily detected in samples treated with 100% PBS positive control, indicating successful transduction and expression from the lentivirus-provided transgene in transduced cells. However, low RLU measurements were obtained in samples treated with as little as 0.1% bile, demonstrating that, even at low concentrations, the presence of bile is detrimental to successful transduction with lentiviral vectors. These data indicate that transduction of target cells with lentiviral vectors is a bile-labile process, and clearly show the negative impact that the presence of bile can have on therapeutic biologics delivered to locations where bile is present.

### Example 2: Bile Acid Sequestrants Rescue Lentiviral Transduction in the Presence of Bile

Bile acid sequestrants, also referred to as bile acid resins, are a class of antilipemic agents that bind to bile acids in the intestine, inhibiting bile acid lipid solubilizing activity and thus blocking cholesterol absorption and inhibiting bile acid reabsorption, which causes a contraction of the bile acid pool and subsequently increased bile acid synthesis that competes with cholesterol synthesis in the liver. This process ultimately results in the lowering of serum cholesterol levels. Various bile acid sequestrants are available for clinical use. All are administered by the oral route exclusively.

Bile acid sequestrants were investigated as candidate reagents to improve transduction of lentiviral vectors in the presence of bile. In this example, 60 μg/mL of sequestrant, colesevelam or colestyramine, was added to mixtures containing 100% PBS, 99.9% PBS/0.1% bile, 99.7% PBS/0.3% bile, 99% PBS/1% bile, 97% PBS/3% bile, 90% PBS/10% bile, or 70% PBS/30% bile and the mixtures were incubated for 30 min at RT. Lentiviral vector, as described above, was added to each mixture and the mixtures were incubated for 1 hour at 37 deg. C. For viability assessment, corresponding mixtures of sequestrant and bile were similarly incubated for 30 min. at RT and then for 1 hour at 37 deg. C. but without addition of lentiviral vector. Following incubation, the solutions were added to cells, with cell media and transduction enhancer, and the cells were transduced/incubated for 72 hours. ONE-glo and CellTiter-Glo assays were used to assess transduction and viability, respectively, as described above.

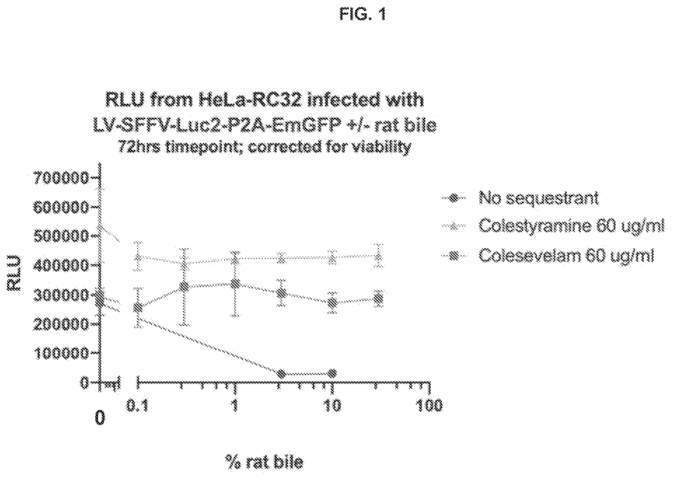

Results of the viability assay are provided in Table 1. These results demonstrate that the presence of sequestrant improves the viability of cells treated with bile. For analysis of the effect of sequestrants on transduction efficiency in the presence bile, luciferase levels were measured in HeLa-RC32 cells after 72 hours of treatment with LV-SFFV-Luc2-P2A-EmGFP in the presence of various bile concentrations as described with or without sequestrant. These measurements were corrected for cell viability and the results of this analysis are provided in FIG. 1.

TABLE 1

| | Percent viability vs. control (average) | | |
|---|---|---|---|
| Bile (%) | No Sequestrant | Colesevelam | Colestyramine |
| 30 | 8.0 | 5.7 | 4.4 |
| 10 | 19.1 | 28.3 | 35.8 |
| 3 | 42.3 | 54.3 | 67.9 |
| 1 | 49.4 | 53.7 | 70.9 |
| 0.3 | 35.5 | 39.9 | 60.7 |
| 0.1 | 33.2 | 49.5 | 66.1 |

As shown, in the absence of bile (0% rat bile) all conditions tested demonstrated efficient transduction of lentiviral vector. In the absence of sequestrant (“No sequestrant”), greatly reduced levels of transduction, approaching a complete lack of transduction, were observed at bile levels above 1%, also indicating that greater than 60% of transduction was lost at bile concentrations around 1% bile. However, addition of either sequestrant, colesevelam or colestyramine, rescued transduction, achieving viability-adjusted luciferase levels at 1% bile that were comparable to the levels observed in the 0% bile condition. Moreover, both sequestrants unexpectedly rescued transduction at bile levels above 1%, even achieving efficient transduction at 30% bile, the highest bile level tested. The bile acid sequestrant, sevelamer, was tested separately using similar methods and also showed an enhancement of transduction as compared to the level of transduction observed with bile in the absence of sequestrant.

Collectively, these data demonstrate that the addition of bile acid sequestrants to transduction compositions where bile is present effectively rescues lentiviral transduction that would be otherwise reduced or ablated by even low amounts of bile. Accordingly, this work shows that the use of bile acid sequestrants enhance the resulting activity of a bile-labile biologic when such sequestrants are included in delivery compositions or therapeutic methods targeted to locations where bile is present.

### Example 3: Sequestrants and Transduction Enhancers Individually Enhance and Rescue Lentiviral Transduction in the Presence of Bile

The prior examples employed transduction enhancer in the transduction media. To better evaluate the transduction-enhancing effect of bile acid sequestrant in bile-containing media, the roles of sequestrant and transduction enhancer were evaluated separately. Briefly, transduction of HeLa-RC32 cells with LV-SFFV-Luc2-P2A-EmGFP vector was performed for 72 hours essentially as described above with transduction enhancer (F108, 1000 μg/mL), sequestrant (colesevelam, 250 μg/mL), or transduction enhancer and sequestrant combination (1000 μg/mL F108+250 μg/mL colesevelam) in a bile dilution series spanning 0.1% to 100% rat bile. The additives, transduction enhancer and/or sequestrant, were co-formulated with vector and incubated for 60 min. prior to transduction. Controls, including a no (0%) bile control and a “no additive” (i.e., no sequestrant or transduction enhancer) control were also evaluated. Quantitation was performed essentially as described herein and measured values were corrected for cell viability.

The results demonstrated that bile acid sequestrant provides significantly increased transduction of lentivirus vector in the presence of various bile concentrations as compared to transduction reactions that contained bile but neither sequestrant nor transduction enhancer. Specifically, the “no additive” control showed reduced levels of transfection with increasing levels of bile whereas, the addition of the transduction enhancer and sequestrant combination resulted in increased transfection levels at all bile dilutions ranging from zero to 100%, thus reinforcing the findings described in the forgoing examples.

Furthermore, the bile acid sequestrant composition that did not contain transduction enhancer nonetheless showed enhanced transduction at various bile levels as compared to the “no additive” control. Accordingly, this example showed that the transduction enhancing effect of sequestrant on bile-labile lentivirus is not dependent on the presence of transduction enhancer. Rather, sequestrant alone demonstrated enhanced transduction in the presence of bile.

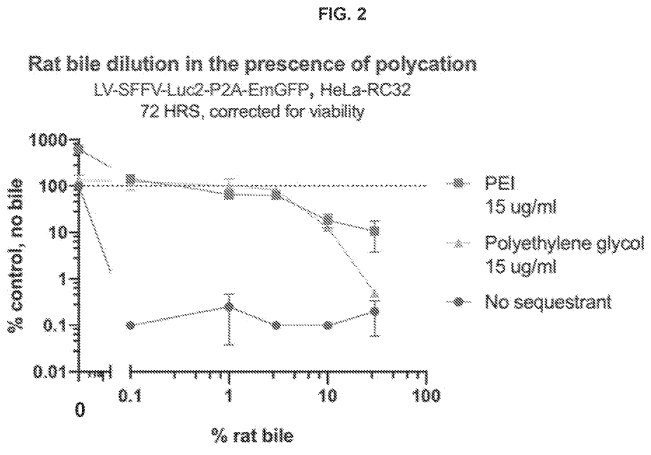

In addition, transduction enhancer F108 was unexpectedly found to rescue transduction even in the absence of sequestrant across various bile concentrations, indicating that transduction enhancers alone can also recover the otherwise bile-labile activity of lentiviral vectors. Other transduction enhancers were also tested. Despite the detrimental effects of even bile on lentivirus transduction as demonstrated herein, other transduction enhancers were also found to have positive effects on the transduction of LV-SFFV-Luc2-P2A-EmGFP vector in the presence of concentrations of bile at and above 1%. For example, as shown in FIG. 2, transduction compositions containing polyethylenimine (PEI, 15 μg/mL) or polyethylene glycol (PEG, 15 μg/mL) rescued vector transduction at rat bile concentrations up to and including 10%, as measured by viability-corrected luciferase expression in transduced HeLa-RC32 cells after 72 hours of transduction with vector. In comparison, negative control, which contained no transduction enhancer (“no transduction enhancer”), demonstrated greatly reduced levels of transduction in the presence of bile at all tested concentrations.

Collectively, these results demonstrate that transduction enhancers and bile acid sequestrants, either individually or in combination, can rescue transduction and enhance the activity of bile-labile lentiviral vectors when bile is present in the transduction environment. This finding was particularly unexpected given the profound impact even small amounts of bile (e.g., less than 1%) were seen to have on the ability of lentivirus to transduce target cells and resulting expression of reporter transgene.

### Example 4: Dynamics of the Inactivation of Lentiviral Vector and Rescue of Vector Activity in the Presence of Human Bile

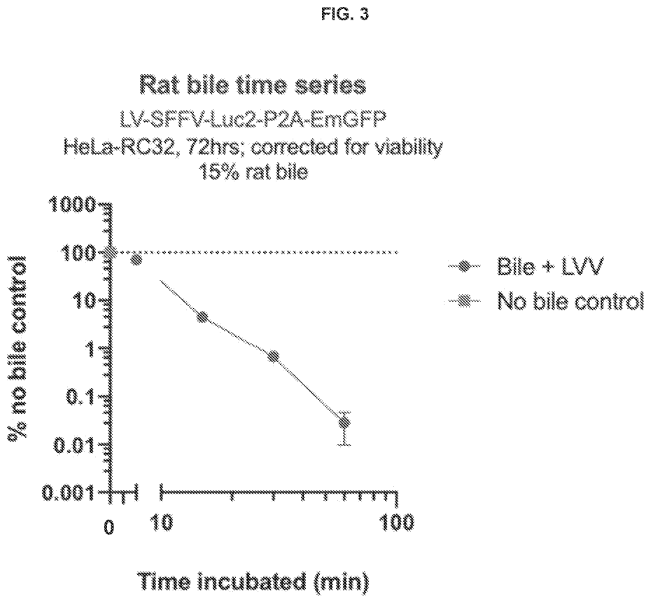

To assess the temporal dynamics of lentiviral vector exposure to bile, a bile-exposure time series was performed. Specifically, LV-SFFV-Luc2-P2A-EmGFP vector was exposed to 15% bile, prepared from a new lot of rat bile, in culture media containing 500 μg/mL F108 for various periods of time ranging from 1 min. to 1 hour. Following incubation of vector in the bile-media solution for the designated amount of time, HeLaRC32 cells were transduced for 72 hours and the resulting impact of the different bile exposure times on the transduction efficiency of the vector was assessed by measuring luciferase reporter transgene as described herein. FIG. 3 provides results of this bile exposure time series with vector activity represented as percentages of the vector activity observed in the absence of bile (“No bile control”) corrected for viability. As shown, after 15 min of exposure to 15% bile, 97% of vector activity was lost and over 99% of activity was lost when bile exposure times were increased to periods above 15 min. In contrast, only about 5% of activity was lost when the vector was exposed to 15% bile for 1 min.

These findings demonstrate that inactivation of lentiviral vector by bile occurs rapidly but also successively over time, adding to the aforedescribed findings that increasing concentrations of bile result in decreasing vector activity. These findings highlight the opportunity to optimize transduction compositions, as well as treatment times, and other transduction parameters to achieve enhanced transduction in environments where bile is present.

In addition to optimizing transduction compositions and other transduction parameters, bile-containing environments may also be modulated in vivo to affect the amount of bile present locally during viral transduction performed within a subject. For example, before delivery of a transduction composition to the bile duct, the duct may be flushed to reduce the concentration of bile within the local environment, thus allowing for the subsequent introduction of transduction composition containing viral vector into a low-bile setting. However, at least because bile is continually produced, it remains unlikely that bile duct flushing, for example, can render the duct completely devoid of bile in a living subject.

In view of the above, the activity of lentiviral vector was evaluated in a human bile dilution series. Briefly, HeLa-RC32 cells were transduced with LV-SFFV-Luc2-P2A-EmGFP vector for 72 hours in the presence of various concentrations of bile ranging from less than 0.01% to more than 10% and vector activity was assessed as a viability-corrected percentage of the reporter measured in controls containing no bile. Human bile levels at and above 10% were observed to be toxic to the cells. However, the results indicated that workable windows, where the enhancements described herein can provide for efficient transduction, exist at human bile levels below 10% that are achievable in the in vivo setting.

Transductions were performed to evaluate the ability of bile acid sequestrant or transduction enhancer to enhance the activity of lentivirus vector in the presence of human bile concentrations of 1% and 0.1%. In brief, LV-SFFV-Luc-P2A-EmGFP vector was incubated with various concentrations of colesevelam or F108 (ranging from less than 10 μg/mL to more than 1000 μg/mL final in-well concentrations) in the respective human bile concentration for 1 hour. Transduction of HeLa-RC32 cells were then performed and vector activity was assessed by reporter expression at 72 hours. Results were reported and compared as viability-corrected reporter activity expressed as a percentage of that measured in control transductions with vector not exposed to bile. The results demonstrated that increasing amounts of either sequestrant or transduction enhancer both enhanced and at least partially rescued lentiviral vector activity after exposure to 0.1% or 1% human bile.

Collectively, these findings indicate that what has been observed with rat bile is transferable to the human bile setting and further demonstrate that bile acid sequestrants and transduction enhancers, both individually and in combination, function to enhance the activity of bile-labile therapeutics, such as lentiviral vectors, in contexts where bile is present. Accordingly, the examples provided herein show that a biologic that would normally be inactivated in the presence of bile may be effectively administered into environments where bile is present, such as the biliary tract, when such administration also employs reagents, such as bile acid sequestrants and/or transduction enhancers, which reduce the detrimental effects of bile on the biologic and/or otherwise enhance the biologic's activity in the presence of bile.

### Example 5: Bile Acid Sequestrant and Transduction Enhancer Formulations Improve Transfection of Cells with Bile-Labile Nonviral Vector

The effect of bile on nonviral vectors was also assessed. Specifically, HeLa-RC32 cells were plated overnight in 24-well plates at a density of 40,000 cells/well. Lipid nanoparticle (LNP) nonviral vector containing EGFP mRNA were pre-incubated with a solution of 30% rat (Sprague Dawley) bile in PBS, or a positive control solution containing PBS without bile, at 37 deg. C. for one hour. The mixtures were then diluted 10× in cell media and cells were incubated with the vector-bile solution at 37 deg. C. with 5% CO2 for 24 hours. Accordingly, cells were exposed to a final bile concentration of 3% in the test group. Following incubation, EGFP expression and cell viability were assessed by flow cytometry.

In the positive control, 90% of cells showed EGFP expression, indicating successful transfection and EGFP expression from the introduced mRNA. However, the cells treated with LNP vector that was exposed to 30% bile showed a 100% loss of LNP-driven EGFP fluorescence as compared to the positive control, indicating a complete impairment of the activity of the LNP EGFP mRNA vector by the presence of bile. As such, these data demonstrate that the activity of nonviral vectors, such as LNP vectors, is liable to degradation in the presence of bile.

The ability of a bile acid sequestrant and transduction enhancer combination to increase the activity of nonviral vector in the presence of bile was also assessed. Specifically, in parallel with the assay described above, LNP nonviral vectors containing EGFP mRNA were pre-incubated with a solution of 30% rat bile and a formulation containing bile acid sequestrant and transduction enhancer (250 μg/mL colesevelam and 10 mg/mL F108 in DPBS) at 37 deg. C. for one hour. As above, the mixture was then diluted 10× in cell media (resulting in final concentrations of 3% bile, 25 μg/mL colesevelam and 1 mg/mL F108) and cells were incubated with the vector-bile solution at 37 deg. C. with 5% CO2 for 24 hours. The cells were then assessed, for EGFP expression and cell viability by flow cytometry, and the results were compared to those obtained from cells treated with bile-exposed LNP as described above.

The sample treated with sequestrant and transduction enhancer showed an unexpected increase of EGFP expression, indicating successful transfection and partial rescue of LNP vector activity lost in the presence of bile. Specifically, 11% of cells treated with media containing LNP, bile, sequestrant, and transduction enhancer showed LNP-driven EGFP expression, as compared to the complete lack of LNP-driven EGFP expression observed in the corresponding sample that contained bile but did not contain sequestrant and transduction enhancer.

Collectively, these results demonstrate that bile sequestrant and transduction enhancer can rescue transfection of nonviral vector and increase the activity of bile-labile nonviral vectors when the vector is exposed to bile and/or bile is present in the transfection environment. This finding was particularly unexpected given the profound impact of bile on LNP vector, resulting in complete impairment of the activity of a vector that otherwise resulted in 90% transfection and expression in the positive control.

### Example 6: Bile Acid Sequestrant and Transduction Enhancer Formulations Individually Improve the Efficiency of LNP-Mediated Transduction in the Presence of Bile

The influence of individual formulation components on transduction of non-viral vectors in the presence of bile was assessed. Amounts of individual formulation components, sequestrant colesevelam or transduction enhancer F108, were titrated in LNP-mediated transduction reactions of GFP encoding nucleic acid essentially as described in Example 5.

HeLa-RC32 cells were plated overnight in 24-well plates at a density of 40,000 cells/well. Transduction reactions of GFP-encoding mRNA in LNPs were performed for 30 min at 37° C. in the presence of 30% rat bile alone (“Bile”), bile and colesevelam (“Bile+colesevelam”), or bile and F108 (“Bile+F108”) at various concentrations. LNP transduction in the absence of bile was also performed as a control (“No Treatment”). At 24 hours following the various transduction reactions, GFP expression was assayed by flow cytometry.

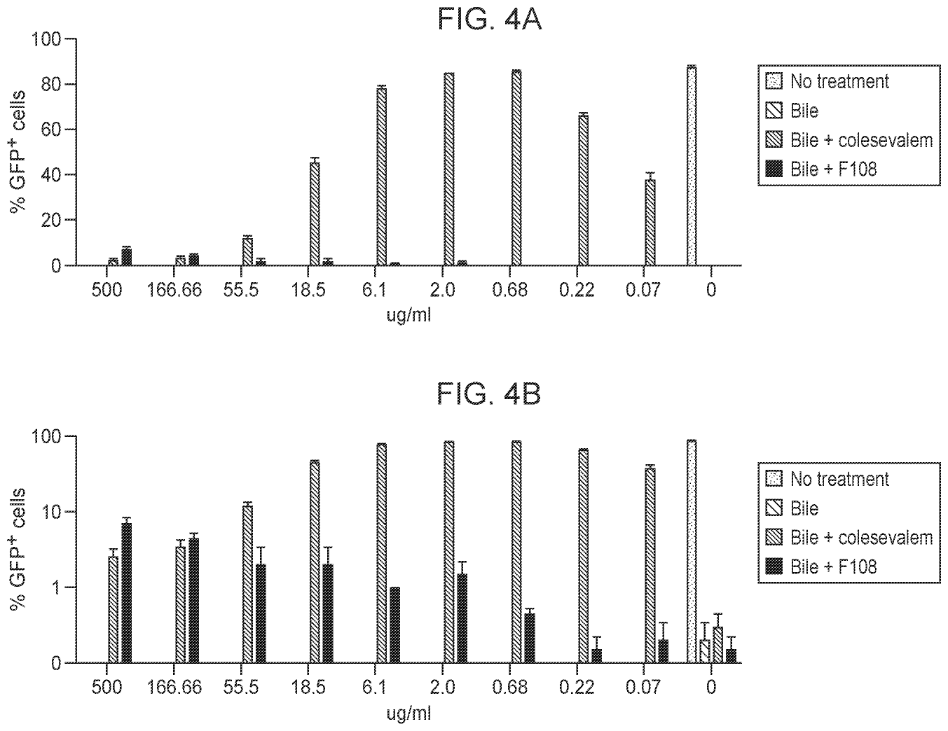

As readily seen in FIG. 4A, sequestrants improved LNP transduction as evidenced by the increased numbers of GFP expressing cells as compared to the numbers of GFP expressing cells seen in bile-containing transduction reactions that did not include sequestrant. Moreover, various colesevelam treatments resulted in near complete rescue of LNP transduction to levels observed in the absence of bile (compare e.g., % GFP+ cells measured in colesevelam concentrations 6.1 μg/mL, 2.0 μg/mL, and 0.68 μg/mL to the % GFP+ cells measured in the “No Treatment” control).

FIG. 4B provides a log-scale rendering of the data presented in FIG. 4A. As can be seen in FIG. 4B, the presence of the F108 enhancer in transduction reactions also improved transduction efficiency in the presence of bile as compared to transduction efficiency observed in the presence of bile without addition of F108.

This example demonstrates that the inclusion of transduction enhancer and/or bile acid sequestrant in transduction reactions where bile is present each individually improve transduction efficiency of non-viral vectors and that the addition of sequestrant can rescue transduction efficiency to levels observed when transduction is performed in the absence of bile.

### Example 7: In Vivo Marking of Liver Following Intraductal Delivery of Lentiviral Vector and Biliary-Therapeutic Enhancer Formulations

Marking of liver cells via intraductal delivery of luciferase-encoding LVV in a bile acid sequestrant and transduction enhancer containing composition was assessed. Briefly, C67/B16 mice surgically implanted with an externalized bile duct catheter were purchased from a commercial vendor. Bile ducts were subjected to retrograde injection according to standard techniques with 150 μL of transduction composition containing diluent containing 7.5E7 TU/mouse lentiviral vector carrying a luciferase expression cassette (i.e., a ubiquitous constitutive promoter such as the synthetic MND promoter operably linked to a sequence encoding luciferase), bile acid sequestrant, and transduction enhancer at final concentrations of μg/mL colesevelam and 500 μg/mL F108. Infusion was performed over 30 seconds. Following infusion the injection syringe was then held in place for 5 minutes to prevent vector efflux. Control animals were infused with a vehicle composition that did not contain the luciferase expression construct-carrying vector. After treatment animals were maintained under standard conditions.

Infused mice were necropsied 4 days after LVV- or vehicle-infusion. Liver samples from left, middle, and right lobes were collected from necropsied animals and fixed in 10% neutral-buffered formalin for 24-48 hrs and then transferred to 70% ethanol. Blocked tissue was sectioned and 3,3′-diaminobenzidine (DAB) staining immunohistochemistry was performed using an anti-luciferase antibody to detect the presence of luciferase expressed by transduced cells.

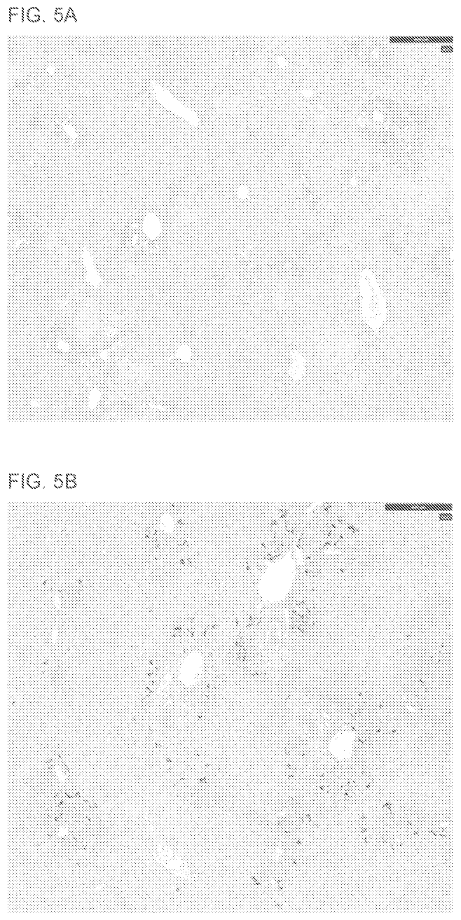

FIGS. 5A and 5B provide representative sections of liver from vehicle and vector (LVV-Luciferase) treated animals, respectively. The presence of DAB positive cells (dark brown) in FIG. 5B demonstrates the successful transduction and expression of luciferase in liver cells of mice that were intraductally administered a composition containing luciferase-encoding LVV, bile acid sequestrant, and transduction enhancer. Positive cells were not detected in control tissues as shown in FIG. 5A.

This example demonstrates the successful in vivo transduction of liver cells via intraductal LVV delivery, and expression of delivered transgene, using a described composition containing bile acid sequestrant and transduction enhancer. Using a delivery composition of the present disclosure, liver cells in living animals were successfully transduced with a bile-labile vector despite the presence of bile in the employed route of delivery.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of the present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of treating a subject for a condition, the method comprising:
administering directly to the biliary tract of the subject an effective amount of a therapeutic biologic and an effective amount of a biliary-therapeutic enhancer thereby treating the subject for the condition.

2. The method of claim 1, wherein the condition is a liver condition.

3. The method of claim 2, wherein the liver condition is selected from the group consisting of: acute intermittent porphyria, acute liver failure, alagille syndrome, alcoholic fatty liver disease, alcoholic hepatitis, alcoholic liver cirrhosis, alcoholic liver disease, alpha 1-antitrypsin deficiency, amebic liver abscess, autoimmune hepatitis, biliary liver cirrhosis, budd-chiari syndrome, chemical and drug induced liver injury, cholestasis, chronic hepatitis, chronic hepatitis b, chronic hepatitis c, chronic hepatitis d, end stage liver disease, erythropoietic protoporphyria, fascioliasis, fatty liver disease, focal nodular hyperplasia, hepatic echinococcosis, hepatic encephalopathy, hepatic infarction, hepatic insufficiency, hepatic porphyrias, hepatic tuberculosis, hepatic veno-occlusive disease, hepatitis, hepatocellular carcinoma, hepatoerythropoietic porphyria, hepatolenticular degeneration, hepatomegaly, hepatopulmonary syndrome, hepatorenal syndrome, hereditary coproporphyria, liver abscess, liver cell adenoma, liver cirrhosis, liver failure, liver neoplasm, massive hepatic necrosis, non-alcoholic fatty liver disease, parasitic liver disease, peliosis hepatis, porphyria cutanea tarda, portal hypertension, pyogenic liver abscess, reye syndrome, variegate porphyria, viral hepatitis, viral hepatitis a, viral hepatitis b, viral hepatitis c, viral hepatitis d, viral hepatitis e, and zellweger syndrome.

4. The method of any of the preceding claims, wherein the administering comprises retroductal delivery of the therapeutic biologic to the liver of the subject thereby resulting in an increase in local hepatic concentration of the therapeutic biologic.

5. The method of any of the preceding claims, wherein the therapeutic biologic comprises a gene therapy agent or a protein.

6. The method of claim 5, wherein the gene therapy agent comprises a nonviral vector or a viral vector, optionally wherein the gene therapy agent comprises a lipid nanoparticle of an enveloped viral vector.

7. The method of any of the preceding claims, wherein the biliary-therapeutic enhancer comprises one or more bile acid sequestrants.

8. The method of claim 7, wherein the one or more bile acid sequestrants comprise a compound selected from the group consisting of:
a compound of formula (I): 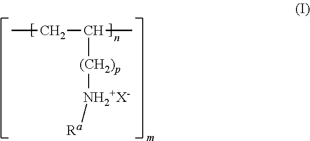
wherein,
m and n correspond to a MW of about 500 to about 1000,
p is an integer from 1 to 3,
X is an electrophilic leaving group,
R^{a} is selected from the group consisting of hydrogen, C₁-C₂₀ alkyl, C₂-C₆ alkenyl, C₂-C₆ alkynyl, or C₁-C₂₀ alkylammonium group, and
wherein the alkyl is optionally substituted with OH or alkylammonium;
a compound of formula (II): 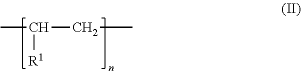
wherein,
n corresponds to a MW of about 100 to about 500, and
R¹ is selected from the group consisting of C₆-C₁₀ aryl or C₂-C₁₀ heteroaryl; wherein the aryl, or heteroaryl, is optionally substituted with 1-3 substituents selected from the group consisting of C₁-C₆ alkyl, C₂-C₆ alkenyl, C₂-C₆ alkynyl, or alkylmmonium halide group;
a compound of formula (V): 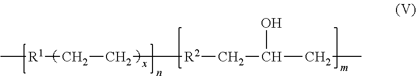
wherein,
m and n correspond to a MW of about 100 to about 500, and
each of R¹ and R² is independently selected from the group consisting of amino, alklyamino or alkylmmonium halide group; and
a compound of formula (VII): 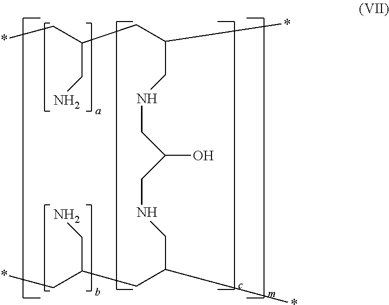
wherein,
m corresponds to a MW of about 100 to about 500,
a is an integer from 1 to 6,
b is an integer from 1 to 6, and
c is an integer from 1 to 3.

9. The method of claim 7 or claim 8, wherein the one or more bile acid sequestrants are selected from the group consisting of colesevelam, colestyramine, colestipol, and sevelamer.

10. The method of any of the preceding claims, wherein the biliary-therapeutic enhancer comprises a cationic or nonionic amphiphilic transduction enhancer.

11. The method of claim 10, wherein the cationic or nonionic amphiphilic transduction enhancer is a compound selected from the group consisting of:
a compound of formula (III): 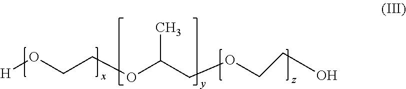
wherein,
each of x, y and z is independently an integer from 1 to 250;
a compound of formula (IVa) or (IVb): 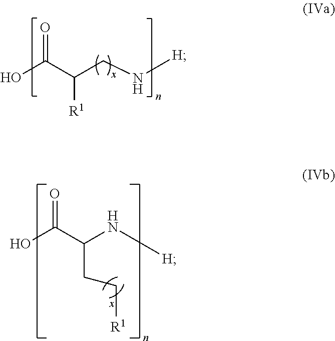
wherein,
n corresponds to a MW of about 50000 to about 200000,
x is an integer from 1 to 6, and
R¹ is an amino group, which is optionally substituted with one or more C₁-C₆ alkyl groups; and
a compound of formula (VI): 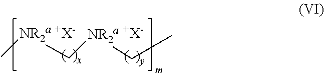
wherein,
m corresponds to a MW of about 100 to about 600,
x is an integer from 1 to 10,
y is an integer from 1 to 6,
X is an electrophilic leaving group, and
each R^{a} is independently a hydrogen or a C₁-C₆ alkyl group.

12. The method of claim 10 or claim 11, wherein the cationic or nonionic amphiphilic transduction enhancer is selected from the group consisting of: polybrene, protamine sulfate, polyethyleneimine (PEI), Poly(ethylene glycol) (PEG), poly-L-lysine, and F108.

13. The method of any of the preceding claims, wherein the therapeutic biologic and the biliary-therapeutic enhancer are co-administered, optionally wherein the therapeutic biologic and the biliary-therapeutic enhancer are co-formulated in a single pharmaceutical composition.

14. The method of any of the preceding claims, wherein the biliary-therapeutic enhancer is administered before the therapeutic biologic.

15. A pharmaceutical composition comprising:
a pharmaceutically acceptable carrier configured as a liquid for delivery to the biliary tract;
an effective amount of a therapeutic biologic; and
a biliary-therapeutic enhancer.

16. The composition of claim 15, wherein the therapeutic biologic comprises a gene therapy agent or a protein, optionally wherein the gene therapy agent comprises a nonviral vector or a viral vector, optionally wherein the gene therapy agent comprises a lipid nanoparticle of an enveloped viral vector.

17. The composition of claim 15 or claim 16, wherein the biliary-therapeutic enhancer comprises a bile acid sequestrant optionally wherein the bile acid sequestrant is a compound selected from the group consisting of:
a compound of formula (I): 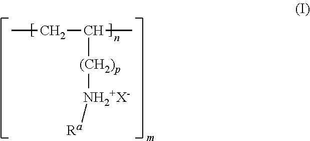
wherein,
m and n correspond to a MW of about 500 to about 1000,
p is an integer from 1 to 3,
X is an electrophilic leaving group,
R^{a} is selected from the group consisting of hydrogen, C₁-C₂₀ alkyl, C₂-C₆ alkenyl, C₂-C₆ alkynyl, or C₁-C₂₀ alkylammonium group, and
wherein the alkyl is optionally substituted with OH or alkylammonium;
a compound of formula (II): 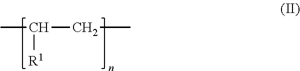
wherein,
n corresponds to a MW of about 100 to about 500, and
R¹ is selected from the group consisting of C₆-C₁₀ aryl or C₂-C₁₀ heteroaryl; wherein the aryl, or heteroaryl, is optionally substituted with 1-3 substituents selected from the group consisting of C₁-C₆ alkyl, C₂-C₆ alkenyl, C₂-C₆ alkynyl, or alkylmmonium halide group;
a compound of formula (V): 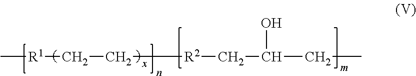
wherein,
m and n correspond to a MW of about 100 to about 500, and
each of R¹ and R² is independently selected from the group consisting of amino, alklyamino or alkylmmonium halide group; and
a compound of formula (VII): 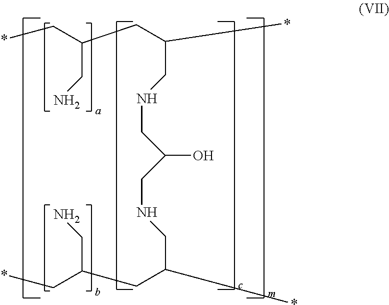
wherein,
m corresponds to a MW of about 100 to about 500,
a is an integer from 1 to 6,
b is an integer from 1 to 6, and
c is an integer from 1 to 3.

18. The composition of any of claims 15 to 17, wherein the bile acid sequestrant is selected from the group consisting of colesevelam, colestyramine, colestipol, and sevelamer.

19. The composition of any of claims 15 to 18, wherein the biliary-therapeutic enhancer comprises a cationic or nonionic amphiphilic transduction enhancer.

20. A kit comprising:
a liquid pharmaceutically acceptable carrier;
a therapeutic biologic; and
a biliary-therapeutic enhancer.
